(19) United States
(12) Patent Application Publication
(10) Pub. No.: US 2024/0374665 A1
(43) Pub. Date: Nov. 14, 2024

(54) CHILLED JUICE EXTRACTION PROCESSES AND PRODUCTS USING PSYCHEDELIC AND FUNCTIONAL MUSHROOMS

(71) Applicant: Joseph Girardi, Hallandale Beach, FL (US)

(72) Inventor: Joseph Girardi, Hallandale Beach, FL (US)

(21) Appl. No.: 18/692,664

(22) PCT Filed: Sep. 28, 2022
(86) PCT No.: PCT/US22/45124

§ 371 Date: Mar. 15, 2024

Related U.S. Application Data
(60) Provisional application No. 63/249,541, filed on Sep. 28, 2021.

(51) Int. Cl. A61K 36/07; A23G 9/36; A23L 2/02; A23L 2/52; A23L 19/00; A23L 29/281; A61K 31/4045; A61K 31/675; A61K 36/068; A61K 36/074; B01D 11/02

(52) U.S. Cl. CPC A61K 36/078; A23G 9/366; A23L 2/02; A23L 2/52; A23L 19/03; A23L 19/09; A23L 29/284; A61K 31/4045; A61K 31/675; A61K 36/068; A61K 36/074; B01D 11/028; B01D 11/0288; A61K 2236/15; A61K 2236/333

(57) ABSTRACT

Disclosed herein are methods of extracting active agents from fungi, in particular psychedelic and functional fungi, and including methods of extracting active agents from fungi using certain natural solvents at particular defined parameters to make extracts and products therefrom having increased palatability and reduced unwanted side effects compared to known extracts and products. Also disclosed are medicinal food and beverage products comprising extracts with one or more active agents obtained using the disclosed methods, as well as methods of their use, such as to produce an effect, or to treat a condition or disorder.

### CROSS-REFERENCE

Priority is claimed under PCT Art. 8(1) and Rule 4.10 to U.S. Appl. No. 63/249,541, filed Sep. 28, 2021, and incorporated by reference for all purposes as if fully set forth herein.

### FIELD OF THE INVENTION

Disclosed are methods of extracting active agents from psychedelic and functional fungi, extracts of active agents from fungi obtained from such methods, products made from such extracts, including food and beverage products, as well as methods of using such products.

### BACKGROUND OF THE INVENTION

Many mushrooms have a strong earthy flavor which many people dislike or even detest, or at the least find distasteful. Consumption of certain mushrooms, especially psychedelic and functional mushrooms, also can upset or unsettle people's stomachs. Some negative side effects may be due to high levels of chitin in fungal cell walls, which is difficult for humans to digest.

Psychedelic and functional mushrooms nonetheless offer many benefits to the human body, mind, and spirit. To combat the above issues, attempts thus have been made to increase the digestibility and palatability of such mushrooms so that their benefits can be enjoyed.

One attempt is “lemon tekking” which describes the technique of soaking mushrooms in lemon juice for a short period of time. Lemon tekking however has several shortcomings. For example, the high level of acidity can cause heartburn and other unpleasant symptoms, and the sour taste of lemon juice can be unpleasant. Thus, new methods of extracting active agents from mushrooms and making products from such extracts, having increased palatability and decreased unpleasant side effects, such as heartburn, nausea, vomiting, and stomach pains, are still needed.

The disclosed invention makes mushrooms easier for people to consume by extracting their beneficial active agents into flavorful juices and then making them into delicious and soothing items like jello, applesauce, rehydrated fruit, popsicles, and ice cubes, which in addition to the other improvements and advantages disclosed herein, make mushrooms tasty, exciting, and fun to consume. As the inventor exclaims, this invention puts the “fun” in fungi!

### INCORPORATION BY REFERENCE

Each patent, publication, and non-patent literature cited in the application is hereby incorporated by reference in its entirety as if each was incorporated by reference individually. Unless specifically stated otherwise, reference to any document herein is not to be construed as an admission that the document referred to or any underlying information in the document is prior art in any jurisdiction, or forms part of the common general knowledge in the art.

### BRIEF SUMMARY OF THE INVENTION

The following presents a simplified summary of some embodiments of the invention in order to provide a basic understanding thereof. This summary is not an extensive overview of the invention. It is not intended to identify key or critical elements of the invention, or to delineate its full scope. Its sole purpose is to present some embodiments of the invention in a simplified form as a prelude to the more detailed description that is presented later.

Active agents produced by psychedelic and functional fungi such as psilocybin, psilocin, norbaeocystin, baeocystin, aeruginascin, norpsilocin, β-carbolines, hericenones, polysaccharides, peptides, triterpenoids, antioxidants, vitamins, and erinacines have many known and potential health benefits. However, processes for obtaining these compounds in extracts and products with a palatable taste are needed. In some aspects are disclosed methods of extracting such active agents from a psychedelic or functional mushroom, the method comprising: obtaining a fungus comprising one or more active agents; preparing the fungus for extraction; obtaining a solvent for extraction; adding the fungus to the solvent and stirring to form a slurry; chilling the slurry for an extraction period; and straining the fungal biomass from the slurry to obtain the extract.

In some embodiments, the solvent is obtained by combining one or more juices with one or more pH modulating ingredients. In some embodiments, the one or more pH modulating ingredients are any of acetic acid, malic acid, apple cider vinegar, and water. In some embodiments, the one or more juices and the one or more pH modulating ingredients are combined to obtain a solvent with a desired pH level. In some embodiments, the solvent does not include any of citric juice and citric acid. In some embodiments, the solvent does not include any of lemon juice, lime juice, and orange juice. In some embodiments, the solvent has a pH of between about 3.0 and 4.0. In some embodiments, the solvent has a pH of greater than 3.5.

In some embodiments, the slurry is continuously stirred throughout the extraction period. In some embodiments, the slurry is periodically stirred throughout the extraction period. In some embodiments, the slurry is periodically stirred separated by intervals in between without stirring of about 1 minute to about 3 days. In some embodiments, the slurry is stirred during the extraction period for at least one stirring period. In some embodiments, the stirring period is from about 1 minute to about 180 minutes. In some embodiments, the extraction period is at least about 4 hours. In some embodiments, the extraction period is for between about 4 to about 130 hours. In some embodiments, the extraction period is for between about 12 to about 120 hours. In some embodiments, the extraction period is for between about 48 to about 110 hours. In some embodiments, the extraction period is for between about 60 to about 96 hours.

In some embodiments, preparing the fungus for extraction includes drying the fungus. In some embodiments, the fungus is dried for about 1 day to about 10 days. In some embodiments, the fungus is cracker dry. In some embodiments, the fungus is pliably dried. In some embodiments, the fungus is pliably dried by drying for about 3 days. In some embodiments, the fungus is dried by using any of a fan, a heat source, and a dehydrator. In some embodiments, preparing the fungus for extraction includes any one or more of chopping, grinding, and pulverizing the fungus. In some embodiments, the stirring is completed by a utensil, such as a spoon, a fork, a knife, and/or a chopstick. In some embodiments, the stirring is completed utilizing an electric stirring device, such as an electric stick blender. In some embodiments, the slurry is chilled for the extraction period in a refrigerator. In some embodiments, the slurry is chilled at a temperature from about 35° F. and about 45° F. In some embodiments, the straining is completed by any of a coffee filter, a cheesecloth, and a muslin cloth.

In some embodiments, the fungus is one or more species within a fungal genera selected from Psilocybe, Agaricus, Pleurotus, Lentinula, Auricularia, Volvariella, Flammulina, Tremella, Hypsizygus, Stropharia, Cyclocybe, Hericium, Phallus, Boletus, Calbovista, Calvatia, Cantharellus, Craterellus, Clitocybe, Cortinarius, Grifola, Macrolepiota, Gyromitra, Hydnum, Lactarius, Morchella, Tricholoma, Tuber, Trametes, Aleuria, Armillaria, Calocybe, Chroogomphus, Clavariaceae, Clavulinaceae, Coprinus, Cortinarius, Cyttaria, Fistulina, Flammulina, Hygrophorus, Kalaharituber, Laetiporus, Leccinum, Macrolepiota, Marasmius, Polyporus, Pseudohydnum, Ramariaceae, Ganoderma, Rhizopogon, Russula, Sparassis, Suillus, Coprinopsis, Lepista, Morchella, Chaga, Cordyceps, Verpa, Copelandia, Galerina, Gymnopilus, Inocybe, Panaeolus, Pholiotina, Inonotus, and Pluteus. In some embodiments, the fungus is a psilocybin mushroom species within a fungal genera selected from a group consisting of Copelandia, Conocybe, Gymnopilus, Galerina, Inocybe, Panaeolus, Pholiotina, Pluteus, and Psilocybe. In some embodiments, the fungus is a lion's mane mushroom. In some embodiments, the fungus is a cordyceps mushroom. In some embodiments, the fungus is a reishi mushroom. In some embodiments, the fungus is a psychedelic or functional mushroom.

In some aspects are provided extracts of a fungus obtained by any of the foregoing methods comprising at least one active agent from a mushroom. In some embodiments, the extract comprises an active agent from a cordyceps mushroom. In some embodiments, the active agent from a cordyceps mushroom is any of cordycepin, cordycepic acid, N-acetylgalactosamine, adenosine, ergosterol, an ergosteryl ester, a bioxanthracene, hypoxanthine, a macrolide, a cicadapeptin, myriocin, superoxide dismutase, naphthoquinone, cordyheptapeptide, dipicolinic acid, a fibrinolytic enzyme, and cordymin. In some embodiments, the extract comprises an active agent from a lion's mane mushroom. In some embodiments, the active agent from a lion's mane mushroom is any of Hericenone A, Hericenone B, Hericenone C, Hericenone D, Hericenone E, Hericenone F, Hericenone G, Hericenone H, Hericenone I, Hericenone J, Hericenone K, 3HF, DLPE, Isohericerinol A, Hericerin, NDPIH, Erinacine A, Erinacine B, Erinacine C, Erinacine D, Erinacine E, Erinacine F, Erinacine G, Erinacine H, Erinacine I, Erinacine J, Erinacine K, Erinacine P, Erinacine Q, and Corallocin A. In some embodiments, the extract comprises an active agent from a psilocybin mushroom. In some embodiments, the psilocybin mushroom is a species within a fungal genera selected from a group consisting of Copelandia, Conocybe, Gymnopilus, Galerina, Inocybe, Panaeolus, Pholiotina, Pluteus, and Psilocybe. In some embodiments, the active agent from a psilocybin mushroom is any of psilocybin, psilocin, norbaeocystin, baeocystin, aeruginascin, norpsilocin, and a β-carboline. In some embodiments, the extract comprises an active agent from reishi. In some embodiments, the active agent from a reishi is any of polysaccharides, triterpenoids, ganodermic acids, lucidenic acids, beta glucans, proteins, amino acids, ganoderic acids, ganodermanontriol, ganodermadiol, palmitic acid, ergosterol, enzymes, steroids, sterols, nucleotides, fatty acids, vitamins and minerals.

In some embodiments, a base is added to the extract to increase the pH of the extract. The extract of claim 46, wherein the base is any of calcium hydroxide, sodium bicarbonate, calcium acetate, and magnesium hydroxide. In some embodiments, the extract further comprises one or more additional ingredients selected from any of: ginger, peppermint, spearmint, cinnamon, Vitamin C, chamomile, licorice root, cardamom, dandelion, marshmallow root, and slippery root.

In some embodiments, the extract or product is formulated in a unit dosage form. In some embodiments, the extract or product comprises a plurality of doses. In some embodiments, the potency of the active agent is less than about 0.05%. In some embodiments, the potency of the active agent is at least about 0.05%. In some embodiments, the potency of the active agent from about 0.05% to about 2.0%. In some embodiments, the active agent is from a psilocybin mushroom, and the potency of the active agent is at least about 0.05%, or from about 0.05% to about 2.0%. In some embodiments, the active agent is from a lion's mane mushroom, and the potency of the active agent is at least about 0.05%, or from about 0.05% to about 2.0%. In some embodiments, the active agent is from a cordyceps mushroom, and the potency of the active agent is at least about 0.05%, or from about 0.05% to about 2.0%. In some embodiments, the active agent is from a reishi mushroom, and the potency of the active agent is at least about 0.05%, or from about 0.05% to about 2.0%.

In some aspects is provided a product made with any of the disclosed extracts, wherein the product is prepared for ingestion. In some embodiments, the product is prepared for ingestion as a popsicle, made by pouring the extract into a mold, inserting a popsicle stick into the mold, and freezing the extract. In some embodiments, the product is prepared for ingestion as an ice cube, made by pouring the extract into a mold and freezing the extract. In some embodiments, the product is prepared for ingestion as a medicinal food or beverage product, made by selecting a food or beverage product, and combining the food or beverage product with the extract. In some embodiments, the product is prepared for ingestion as an apple sauce, made by preparing at least one apple and combining the prepared apple(s) with the extract and optionally with a quantity of a sweetening agent. In some embodiments, the product is prepared for ingestion as a jello, made by heating water until hot, combining the hot water with gelatin powder, mixing the gelatin powder and water until the gelatin powder is dissolved in the water forming a mixture, allowing the mixture to cool to room temperature, adding the extract to the mixture, and chilling the mixture until it forms the jello. In some embodiments, the product is prepared for ingestion by being formulated as a rehydrated fruit, made by combining the extract with dehydrated fruit and allowing the dehydrated fruit to rehydrate by absorbing the extract. In some embodiments, the product is prepared for ingestion as a juice made by combining the extract with at least one juice. In some embodiments, the product further comprises at least one additional ingredient. In some embodiments, the additional ingredient is any of ginger, peppermint, spearmint, cinnamon, Vitamin C, chamomile, licorice root, cardamom, dandelion, marshmallow root, and slippery root.

In some embodiments, the product does not produce at least one negative symptom selected from any of nausea, heartburn, stomach pain, and vomiting. In some aspects, the product is used to enhance any of mood, cognition, relaxation, wellbeing, and sleep.

In some embodiments, the product elicits a therapeutic effect. In some embodiments, the therapeutic effect is any of antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, nootropic, entactogenic, empathogenic, entheogenic, euphoric, psychedelic, sedative, and stimulant effects. In some embodiments, the therapeutic effect is a psychedelic effect.

In some aspects, any of the disclosed products is used to treat a medical condition in a human, such as in any of the disclosed methods. In some embodiments, the medical condition is any of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, a substance use disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders. In some embodiments, the medical condition is a mental health disorder related to rigid modes of thinking. In some embodiments, the disorder related to rigid modes of thinking is any of anxiety, depression, addiction, an eating disorder, an alcohol or drug abuse or dependence disorder, OCD, or PTSD. In some embodiments, depression is major depressive disorder (MDD) or treatment-resistant depression (TRD). In some embodiments, anxiety is generalized anxiety disorder (GAD). In some embodiments, the substance use disorder is any of alcohol use disorder, nicotine dependency, opioid use disorder, sedative, hypnotic, or anxiolytic use disorder, stimulant use disorder, and tobacco use disorder.

In some embodiments, the medical condition is a neurodegenerative disorder. In some embodiments, the neurodegenerative disorder is any of Alzheimer's disease (AD), corticobasal degeneration (CBD), a form of dementia, Huntington's disease, Lytico-Bodig disease, mild cognitive impairment (MCI), a motor neuron disease, progressive supranuclear palsy (PSP), multiple sclerosis, Parkinson's disease, and traumatic brain injury (TBI).

In some embodiments, the medical condition is pain or a pain disorder. In some embodiments, the pain disorder is any of arthritis, allodynia, atypical trigeminal neuralgia, trigeminal neuralgia, somatoform disorder, hypoesthesia, hyperalgesia, neuralgia, neuritis, neurogenic pain, phantom limb pain, analgesia, anesthesia dolorosa, causalgia, sciatic nerve pain disorder, degenerative joint disorder, fibromyalgia, visceral disease, chronic pain disorders, headache disorders, migraine headaches, chronic cluster headaches, concussion headache, short-lasting unilateral neuralgiform headache attacks, chronic fatigue syndrome, complex regional pain syndrome, neurodystrophy, plantar fasciitis, and pain associated with cancer.

In some embodiments, the medical condition is inflammation or an inflammatory disorder. In some embodiments, the inflammatory disorder is characterized by any one or more of skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation, intestinal inflammation, neuroinflammation, and brain inflammation.

In some embodiments, the product is formulated in a unit dosage form. In some embodiments, the product comprises a plurality of doses. In some embodiments, the product comprises a single dose. In some embodiments, the product comprises an active agent from a mushroom in an amount so that a single dose of the active agent is about 1 mg or less including a dose of about 0.5 mg or less, about 0.25 mg or less, about 0.1 mg or less, about 0.05 mg or less, about 0.005 mg or less, about 0.001 mg or less, and about 0.0005 mg or less. In some embodiments, the product comprises an active agent from a mushroom in an amount so that a single dose is at least about 1 mg to about 1000 mg or more. In some embodiments, the active agent is from a psilocybin mushroom in an amount so that a single dose is from about 1 mg to about 70 mg. In some embodiments, the active agent is psilocybin in an amount so that a single dose is from about 1 mg to about 70 mg. In embodiments, the active agent is psilocin in an amount so that a single dose is from about 1 mg to about 70 mg. In embodiments, the active agent is from a lion's mane mushroom in an amount so that a single dose is from about 20 mg to about 500 mg. In embodiments, the active agent is from a cordyceps mushroom in an amount so that a single dose is from about 20 mg to about 500 mg. In embodiments, the active agent is from a reishi mushroom in an amount so that a single dose is from about 20 mg to about 500 mg.

In some embodiments, the product is formulated to comprise a plurality of macrodoses. In some embodiments, the product is formulated to comprise a plurality of microdoses. In some embodiments, the product is administered together with one or more sessions of psychotherapy.

These and other objects, features, improvements, and advantages of the present invention will be more clearly understood and appreciated from a review of the following detailed description of the disclosed embodiments and examples, and by reference to the appended claims. The foregoing summary has been made with the understanding that it is to be considered as a brief and general synopsis of only some of the objects and embodiments disclosed herein, is provided solely for the benefit and convenience of the reader, and is not intended to limit in any manner the scope, or range of equivalents, to which the appended claims are lawfully entitled.

Additional features of the invention will be described hereinafter which also form the subject of the claims. It will be appreciated by those of skill that the conception and disclosed methods and products may be readily utilized as a basis for modifying or designing other methods and products. It should be also realized that such equivalent methods and products do not depart from the spirit and scope of the invention as set forth in the appended claims.

### BRIEF SUMMARY OF THE DRAWINGS

To further clarify various aspects of the invention, a more particular description is rendered by reference to certain exemplary embodiments thereof which are illustrated in the figures. It will be understood and appreciated that these figures depict only illustrated embodiments of the invention and are not to be considered limiting of its scope. They are simply provided as exemplary illustrations of certain concepts of some embodiments of the invention.

Certain aspects are thus further described and explained with additional specificity and detail, but still by way of example only, with reference to the accompanying figures in which:

FIG. 1 is a flowchart of an exemplary process for extracting active agents from fungi, in accordance with an embodiment of the invention.

FIG. 2 is a flowchart of an exemplary process for formulating an extract into a medicinal food or beverage, in accordance with an embodiment of the invention.

FIG. 3 is a flowchart of an exemplary process for formulating an extract into a medicinal ice cube or popsicle, in accordance with an embodiment of the invention.

### DETAILED DESCRIPTION

While the embodiments of the invention are now further described in terms of particular embodiments, examples, and applications, and by reference to the exemplary embodiments that are depicted in the figures, this description is not limited to any such embodiments, examples, and applications, and it will be understood that many modifications, substitutions, alternatives, changes, and variations in the described embodiments, examples, applications, and other details of the invention illustrated herein can be made by those skilled in the art without departing from the spirit of the invention, or the scope of the invention as described in the appended claims, including all equivalents to which they are lawfully entitled. While the methods described and illustrated herein may include particular steps, it should be apparent that other methods including fewer, more, or different steps than those described and shown, and methods providing the particular steps in a different order, are also within the spirit and scope of the invention. The methods and uses of any device or apparatus discussed and associated steps shown herein therefore should be understood as being provided for purposes of illustration, not limitation.

### A. GENERAL DEFINITIONS AND TERMS

When introducing elements of the invention or the embodiments thereof, the articles “a,” “an,” “the,” and “said” are intended to mean that there are one or more of the elements. Any reference to an element in the singular is therefore not intended to mean “one and only one” unless specifically so stated, but rather “one or more”; thus, the term “or” standing alone, unless context demands otherwise, shall mean the same as “and/or.” The terms “comprising,” “including,” “such as,” and “having” are also intended to be inclusive and not exclusive (i.e., there may be other elements in addition to the recited elements). Thus, for example, the terms “including,” “may include,” and “include” mean, and are used interchangeably with, the phrase “including but not limited to.” The word “exemplary” is used to mean “serving as an example, instance, or illustration.” Any aspect, embodiment, process, or implementation described herein as “exemplary” is thus not to be construed as necessarily preferred or advantageous over others.

Unless otherwise stated, all measurements, values, ratings, positions, magnitudes, sizes, locations, orientations, configurations, and other specifications that are set forth (either expressly or impliedly) in this specification, including in the figures and in the claims that follow, are approximate, and not exact. They are intended to have a reasonable range that is consistent with the functions to which they relate and with what is customary in the art to which they pertain. Unless otherwise stated, all numbers expressing quantities of ingredients, properties such as concentration, reaction conditions, and so forth, used to describe and claim certain embodiments of the invention are to be understood as being modified in some instances by the term “about.” Where the term “about” is used to modify a value, it means that a range of values that includes the specified value is also encompassed, which a person of ordinary skill would consider reasonably similar to the specified value. Where numerical values may be modified by the term “about,” it will be understood that in some embodiments, such values are modified by “about,” but in other embodiments, such values are within a further degree of precision. In some embodiments, “about” means within a standard deviation using measurements generally acceptable in the art. In some embodiments, “about” means within known tolerances. In other embodiments, “about” means a range extending to +/−10% of the specified value. Accordingly, in some embodiments, the numerical parameters set forth in the description and claims are approximations that can vary depending upon the desired properties sought to be obtained by a particular embodiment. “Substantially,” when applied to modify a parameter or characteristic herein, will be read in the context of the invention and in light of the knowledge in the art to provide certainty, e.g., by using a standard that is recognized in the art for measuring the meaning of substantially as a term of degree, or by ascertaining the scope as would one of skill.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable, taking into consideration known tolerances. The numerical values presented in some embodiments thus may contain certain errors necessarily resulting from the standard deviation found in their respective testing measurements.

Generally, the nomenclature used and procedures performed herein are those known in field(s) relating to that of one or more aspects of the invention, and are those that will be well-known and commonly employed in such field(s). Standard techniques and procedures are those generally performed according to conventional methods in the art. Unless defined otherwise, all technical and scientific terms have the meaning as commonly understood by a person having ordinary skill in the art to which this invention belongs (“one of skill”).

Further definitions that may assist the reader in understanding the disclosed embodiments are as follows; however, it will be appreciated that they will not be used to limit the scope of the invention, which shall be properly interpreted and understood by reference to the full specification (as well as any plain meaning known to one of skill in the relevant art) in view of the language used in the appended claims. That is, the terminology used herein is for the purpose of describing particular embodiments only and is not intended to be limiting.

“Fungus” or “fungi” will be understood to encompass the fungal species described herein, and all fungal parts from such species, whether usually found above or below ground, such as fruiting bodies (including caps and stipes), mycelia, and sclerotia (“truffles”). “Fungus” or “fungi” may be used interchangeably herein with the term “mushroom.”

Fungal species contemplated for use in the invention include, as non limiting examples, psilocybin mushrooms, lion's mane mushrooms, cordyceps mushrooms, and reishi mushrooms.

In some embodiments, the mushroom is a psilocybin mushroom. “Psilocybin mushroom” refers to any fungus that contains psilocybin. A psilocybin mushroom also may contain other alkaloids such as psilocin, norbaeocystin, baeocystin, aeruginascin, norpsilocin, and β-carbolines. Thus, an active agent from a psilocybin mushroom may be, as examples, any one or more of psilocybin, psilocin, norbaeocystin, baeocystin, aeruginascin, norpsilocin, and β-carbolines. Exemplary psilocybin mushrooms include those from the genera Copelandia, Conocybe, Gymnopilus, Galerina, Inocybe, Panaeolus, Pholiotina, Pluteus, and Psilocybe.

In some embodiments, the mushroom is a lion's mane mushroom. “Lion's mane mushroom” refers to any fungus from the genus Hericium, and in particular those species bearing colloquial names including “Lion's mane,” “monkey head mushroom,” “bearded tooth mushroom,” “satyr's beard,” “bearded hedgehog mushroom,” “pom pom mushroom,” and/or “bearded tooth fungus” (Miller, 1933), as will be known to those in the art. As used herein, “Lion's mane” may refer in preferred embodiments to the species Hericium erinaceus. Other Lion's mane mushrooms include those from the species H. abietis, H. alpestre, H. americanum, H. bharengense, H. botryoides, H. cirrhatum, H. clathroides, H. coralloides, H. novae-zealandiae, H. rajchenbergii, and H. yumthangense. An active agent from a lion's mane mushroom may be, as examples, any one or more hericenones from Hericium erinaceum, and including any of Hericenone A, Hericenone B, Hericenone C, Hericenone D, Hericenone E, Hericenone F, Hericenone G, Hericenone H, Hericenone I, Hericenone J, Hericenone K, 3HF, DLPE, Isohericerinol A, Hericerin, NDPIH, and Corallocin A.

In some embodiments, the mushroom is a cordyceps mushroom. “Cordyceps mushroom” refers to those mushroom species as commonly understood in the art, such as Ophiocordyceps sinensis. An active agent from a cordyceps mushroom may be, as examples, any one or more of cordycepin, cordycepic acid, N-acetylgalactosamine, adenosine, ergosterol, ergosteryl esters, bioxanthracenes, hypoxanthine, acid deoxyribonuclease, polysaccharide, exopolysaccharide, chitinase, macrolides, cicadapeptins, myriocin, superoxide dismutase, protease, naphthoquinone, cordyheptapeptide, dipicolinic acid, fibrinolytical enzyme, lectin, and cordymin.

In some embodiments, the mushroom is a reishi mushroom. “Reishi mushroom” refers to those mushroom species as commonly understood in the art, such as the mushroom species Ganoderma lucidum. An active agent from a reishi mushroom may be, as examples, any one or more of polysaccharides, triterpenoids, ganodermic acids, lucidenic acids, beta glucans, proteins, amino acids, ganoderic acids, ganodermanontriol, ganodermadiol, palmitic acid, ergosterol, enzymes, steroids, sterols, nucleotides, fatty acids, vitamins and minerals.

In some embodiments, the mushroom is a psychedelic mushroom. In some embodiments, the mushroom is a functional mushroom. A “psychedelic mushroom” and a “functional mushroom” will be as generally understood to those of ordinary skill in the art.

In some embodiments, at least one species of fungus selected for extraction is from the genera Psilocybe, Agaricus, Pleurotus, Lentinula, Auricularia, Volvariella, Flammulina, Tremella, Hypsizygus, Stropharia, Cyclocybe, Hericium, Phallus, Boletus, Calbovista, Calvatia, Cantharellus, Craterellus, Clitocybe, Cortinarius, Grifola, Macrolepiota, Gyromitra, Hydnum, Lactarius, Morchella, Tricholoma, Tuber, Trametes, Aleuria, Armillaria, Calocybe, Chroogomphus, Clavariaceae, Clavulinaceae, Coprinus, Cortinarius, Cyttaria, Fistulina, Flammulina, Hygrophorus, Kalaharituber, Laetiporus, Leccinum, Macrolepiota, Marasmius, Polyporus, Pseudohydnum, Ramariaceae, Ganoderma, Rhizopogon, Russula, Sparassis, Suillus, Coprinopsis, Lepista, Morchella, Chaga, Cordyceps, Verpa, Copelandia, Galerina, Gymnopilus, Inocybe, Panaeolus, Pholiotina, Inonotus, and Pluteus.

In some embodiments, the species of fungus selected for extraction is at least one of Psilocybe cubensis, Psilocybe acutipilea, Psilocybe allenii, Psilocybe alutacea, Psilocybe angulospora, Psilocybe antioquiensis, Psilocybe araaucariicola, Psilocybe atlantis, Psilocybe acquamarina, Psilocybe armandii, Psilocybe aucklandiae, Psilocybe aztecorum, Psilocybe baeocystis, Psilocybe caeruleoannulata, Psilocybe caerulescens, Psilocybe caerulipes, Psilocybe callosa, Psilocybe carbonaria, Psilocybe chuxiongensis, Psilocybe collybiodes, Psilocybe columbiana, Psilocybe cordispora, Psilocybe cyanescens, Psilocybe cyanofibrillosa, Psilocybe dumontii, Psilocybe egonii Guzmán, Psilocybe eximia, Psilocybe fagicola, Psilocybe farinacea, Psilocybe fimetaria, Psilocybe fuliginosa, Psilocybe furtadoana, Psilocybe galindoi, Psilocybe gallaeciae, Psilocybe graveolens, Psilocybe guatopenis, Psilocybe guilartensis, Psilocybe heimii, Psilocybe herrerae, Psilocybe hispanica, Psilocybe hoogshagenii, Psilocybe inconspicua, Psilocybe indica, Psilocybe isabelae, Psilocybe jacobsii, Psilocybe jaliscana, Psilocybe kumaenorum, Psilocybe laurae, Psilocybe lazoi, Psilocybe liniformans, Psilocybe mairei, Psilocybe makarorae, Psilocybe mammillata, Psilocybe medullosa, Psilocybe meridensis, Psilocybe meridionalis, Psilocybe mescaleroensis, Psilocybe mexicana, Psilocybe moseri, Psilocybe muliercula, Psilocybe naematoliformis, Psilocybe natalensis, Psilocybe natarajanii, Psilocybe neorhombispora, Psilocybe neoxalapensis, Psilocybe ovoideocystidiata, Psilocybe papuana, Psilocybe paulensis, Psilocybe pelliculosa, Psilocybe pintonii, Psilocybe pleurocystidiosa, Psilocybe plutonia, Psilocybe portoricensis, Psilocybe pseudoaztecorum, Psilocybe puberula, Psilocybe quebecensis, Psilocybe rickii, Psilocybe rostrata, Psilocybe rzedowskii, Psilocybe samuiensis, Psilocybe schultesii, Psilocybe semilanceata, Psilocybe septentrionalis, Psilocybe serbica, Psilocybe sierrae, Psilocybe silvatica, Psilocybe singeri, Psilocybe strictipes, Psilocybe stuntzii, Psilocybe subacutipilea, Psilocybe subaeruginascens, Psilocybe subaeruginosa, Psilocybe subbrunneocystidiata, Psilocybe subcaerulipes, Psilocybe subcubensis, Psilocybe subpsilocybioides, Psilocybe subtropicalis, Psilocybe tampanensis, Psilocybe tasmaniana, Psilocybe thaiaerugineomaculans, Psilocybe thaicordispora, Psilocybe thaiduplicatocystidiata, Psilocybe uruguayensis, Psilocybe uxpanapensis, Psilocybe venenata, Psilocybe wassoniorum, Psilocybe weilii, Psilocybe weldenii, Psilocybe weraroa, Psilocybe xalapensis, Psilocybe yungensis, Psilocybe zapotecoantillarum, Psilocybe zapotecocaribaea, Psilocybe zapotecorum, Pholiotina cyanopus, Pholiotina smithii, Panaeolus venezolanus, Panaeolus tropicalis, Panaeolus tirunelveliensis, Panaeolus rubricaulis, Panaeolus olivaceus, Panaeolus moellerianus, Panaeolus microsporus, Panaeolus lentisporus, Panaeolus fimicola, Panaeolus cyanescens, Panaeolus cinctulus, Panaeolus chlorocystis, Panaeolus cambodginiensis, Panaeolus bisporus, Panaeolus axfordii, Panaeolus africanus, Panaeolus affinis, Pluteus albostipitatus, Pluteus americanus, Pluteus cyanopus, Pluteus glaucus, Pluteus glaucotinctus, Pluteus nigroviridis, Pluteus phaeocyanopus, Pluteus salicinus, Pluteus saupei, Pluteus velutinornatus, Pluteus villosus, Inocybe aeruginascens, Inocybe caerulata, Inocybe coelestium, Inocybe corydalina, Inocybe haemacta, Inocybe tricolor, Gymnopilus aeruginosas, Gymnopilus braendlei, Gymnopilus cyanopalmicola, Gymnopilus dilepis, Gymnopilus dunensis, Gymnopilus intermedius, Gymnopilus lateritius, Gymnopilus luteofolius, Gymnopilus luteoviridis, Gymnopilus luteus, Gymnopilus palmicola, Gymnopilus purpuratus, Gymnopilus subpurpuratus, Gymnopilus subspectabilis, Gymnopilus validipes, Gymnopilus viridans, Conocybe siligineoides, Conocybe velutipes, Copelandia tropica, Galerina steglichii, Hericium erinaceus, Ganoderma lingzhi, Inonotus obliquus, Cordyceps militaris, Lentinula edodes, Trametes versicolor, Grifola frondosa, Flammulina filiformis, and Pleurotus ostreatus.

“Active agent from a mushroom” means any compound obtained from a mushroom, where the compound is capable of producing, increasing, reducing, or otherwise modulating a desired physiological response in mammals, preferably humans. In some preferred embodiments, active agents from mushrooms include psilocybin, psilocin, norpsilocin, baeocystin, norbaeocystin, aeruginascin, hericenones, erinacines, β-carbolines, triterpenoids, and cordycepin.

“Solvent” means one or more fruit or vegetable juices alone, or in combination with other pH modulators such as water, apple cider vinegar, acetic acid, or malic acid. In a preferred embodiment, “solvent” will mean a juice having a pH of about 3.0 to about 4.0.

“Extract” means a filtrate obtained from the disclosed extraction method containing one or more active agents.

“Extraction period” means an amount of time used to extract one or more active agents from a mushroom, including to extract a desired quantity of active agent(s) from a mushroom.

In some aspects, the disclosed extracts and products are used in methods to produce one or more therapeutic effects, which may also include psychedelic effects, in a subject. In some embodiments, the therapeutic effects and/or psychedelic effects are used for treatment.

“Therapeutic effects” include, but are not limited to, antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, nootropic, entactogenic, empathogenic, entheogenic, euphoric, psychedelic, sedative, and stimulant effects. “Psychedelic effects” refer to subjective alterations in perception, cognition, emotion, or consciousness that can affect and usually interfere with the ability to perform everyday tasks and activities. Although a determination of psychedelic effects can be made by a subject following consumption of a composition of the invention, such determination also can be made by use of or by reference to psychometric rating scales such as those known in the art, e.g., the Hallucinogen Rating Scale (HRS), Mystical Experience Questionnaire (MEQ), Addiction Research Center Inventory (ARCI) (see Bouso et al., 2016), and other scales for assessing subjective drug effects.

The terms “subject,” “patient,” and “individual” are used interchangeably, and refer to any mammal, including murines, simians, humans, mammalian farm animals, mammalian sport animals, and mammalian pets. Preferably, the subject herein is human. As further used herein, the terms “subject,” “patient,” and “individual” includes a subject or patient who has a mental health condition, or a condition related to a mental health condition for which similar treatment may be efficacious. Such terms shall also refer to patients in need of treatment for such a disorder, persons predisposed to such a disorder, and subjects who have been diagnosed with such a disorder. Moreover, these terms shall likewise refer to persons who have received treatment or therapy for a mental health condition, are currently receiving therapy or treatment for a mental health condition, or who may receive therapy or treatment for such a disorder in the future. In embodiments, the disclosed methods also can be used to improve mental health and improve psychological functioning in non-disease states, i.e., in an individual without a diagnosed mental disorder, or specific symptoms thereof.

“Treatment” covers any treatment of a disorder in a mammal, and particularly in a human, and includes: (a) preventing a disorder from occurring in a subject who may be predisposed to the disorder but has not yet been diagnosed with it: (b) inhibiting a disorder, i.e., arresting its development; (c) relieving a disorder, i.e., causing regression thereof; (d) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder. Other such measurements, benefits, and surrogate or clinical endpoints, alone or in combination, would be understood to one of ordinary skill.

### B. OVERVIEW

In some aspects are disclosed processes for extracting active agents from fungi. In one example embodiment of the invention, fungi, either fresh, dried or a combination thereof, are prepared in a manner that increases their surface area and are combined with an acidic solvent (i.e., a solvent having a pH<7). The combined mixture of fungi and solvent is then stirred to form a slurry. The slurry is then placed in a temperature controlled environment and allowed to stand for a period of time sufficient to extract active agents from the mushrooms, which herein may be referred to as an “extraction period.” Following the extraction period, the fungal biomass is filtered out of the slurry to obtain an extract, containing at least one active agent.

In embodiments, the extract has a defined potency. Potency can be determined by w/w %, e.g., by dividing the weight of a compound to be assessed with the total weight of an extract, or by w/v % e.g., by dividing the weight of a compound to be assessed with the total volume of an extract. In embodiments, the potency of active agent in an extract is at least about 0.25%, including about 0.25%, 0.50%, 0.75%, 1.0%, 1.1%, 1.2%, 1.3%, 1.4%, 1.5%, 1.6%, 1.7%, 1.8%, 1.9%, 2.0%, and greater than 2.0%, as may be obtained through utilizing the methods of the invention as disclosed herein. In embodiments, potency is equivalent to “concentration.” In embodiments, an extract, which may optionally be further concentrated, is standardized. A “standardized” extract refers to an extract comprising a specified quantity of a standardized ingredient, which may be an active agent such as psilocybin. Thus, in embodiments, an amount of the active agent, such as an amount of psilocybin, is standardized to a particular concentration (e.g., w/w or w/v % of the extract). In embodiments, an extract will be standardized so as to contain by weight percent an amount of an active agent (i.e., mg active agent per mg or mL of extract, depending on whether such extract is a dry powder or a liquid) of 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% w/w or w/v active agent. In embodiments, the extract will contain by weight % an amount of active agent (i.e., mg active agent per mg extract) of 0.05% or less, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, or 0.30% w/w or w/v active agent.

In some embodiments, the extract is standardized to a chosen potency. Standardization may be accomplished by methods known to those in the art, such as measuring a concentration of compound in an extract to be standardized, determining the concentration of the compound to be standardized, determining an amount of excipient necessary to obtain a desired (standardized) concentration, and then adding the amount of excipient necessary to obtain the desired (standardized) concentration, resulting in a standardized extract. An excipient will be as known by ordinary skill, and may be a dry or liquid excipient, to create a dry powder or liquid standardized extract. Optionally, the concentration of standardized compound in standardized extract may be measured after adding one or more portions of excipient or after the final standardized extract is prepared, to confirm the standardization method and for quality control.

In embodiments, the extract is further concentrated so that the active agents are increased in total concentration from an initial extract, such as an increase in w/w % (for a powder extract) or w/v % (for a liquid extract), in an amount such as by at least 5%, at least 10%, at least 15%, at least 20%, at least 25%, at least 30%, at least 40%, at least 45%, at least 50%, at least 60%, at least 70%, at least 80%, at least 90%, at least 100% (2×), at least 125%, at least 150%, at least 175%, at least 200% (3×), at least 250%, at least 300% (4×), at least 400% (5×), at least 500% (6X), at least 600% (7×), at least 700% (8×), at least 800% (9×), at least 900% (10×), and in amounts of 1,000% or more. Methods of concentrating an extract include, e.g., evaporating a portion of the extract to create a volume of concentrated extract at a desired concentration.

In some aspects are disclosed products, such as medicinal food and beverage products, comprising an extract suitable for oral consumption, such as ice cubes, popsicles, apple sauce, jello, and rehydrated fruit. Medicinal food products are produced by combining a food or beverage with one or more extracts containing active agents obtained using the disclosed methods. Contemplated products include any food or beverage which may be infused, mixed or otherwise combined with an extract containing active agents.

In some embodiments, the extracts are formulated in a unit dosage form. The term “unit dosage form” refers to a physically discrete unit suited as unitary dosages for the subject to be treated, each unit comprising a predetermined quantity of active material calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose), of the combinations administered. Unit dosage forms include extracts, ice cubes, beverages, foods, and popsicles.

### C. METHODS OF EXTRACTION

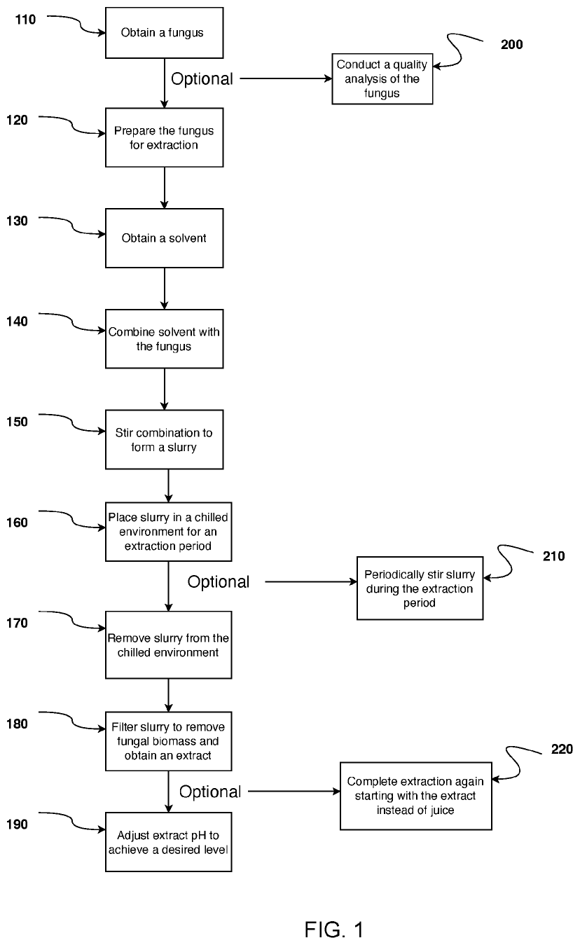

The invention relates to the extraction (FIG. 1) of active agents from at least one fungus. The present extraction methods may be performed on any portion of a fungus containing active agents, including fruiting bodies, sclerotium, mycelium or some combination thereof. The extraction methods may be performed on any fungus containing active agents. In some embodiments, at least one fungus is selected (110) to be used in the extraction process based upon the one or more active agents sought to be extracted. The extraction process may be carried out on more than one fungal species simultaneously to obtain active agents from multiple species in a single extract. Additionally, extracts from multiple extractions performed on several fungal species individually may be combined after extraction to achieve a desired active agent profile.

In embodiments, the extraction comprises a step wherein the fungi are prepared for extraction, various means of preparation will be appreciated by those of skill (120). For example, the fungi may be prepared prior to extraction in a manner that increases the surface area thereof. Additionally, as part of the preparation step, the fungi may be dried (also termed “dehydrated”).

In some embodiments, the fungus utilized in the process of the invention (FIG. 1) is “substantially dry,” having a moisture content of from about 2% to about 3% moisture by weight. That said, in some embodiments, the fungus may have less than 2% or more than 3% moisture by weight. For example, dehydrated mushrooms may have a moisture content of from about 4% to about 7% by weight. If not substantially dry when harvested, the fungus may be dried (120) in a dehydrator or by application of heat in any conceivable method known to those of skill, including but not limited to conduction, convection, or radiation. Dehydration or drying of the fungus may also be achieved by utilization of forced air using any acceptable means of directing an air current, for example with the use of a fan.

It will be appreciated, however, that if dried below 2%, or at too high of a temperature, active agents could be degraded, reducing the final yield of active agent that will be obtained by the process. The appropriate amount of drying may be determined by practice of ordinary skill for the specific embodiment of the invention being carried out. In one illustrative, non-limiting way of determining an adequate level of moisture content, mushrooms in the desired moisture range easily snap in half. So, dehydration is often completed until the fungi are “cracker dry.”

That said, drying the harvested fungus is merely an optional step, as discussed further below. Thus, embodiments wherein the fungi are not dried, or are of a moisture content outside the range disclosed above (e.g., 1%, 2%, 3%, 4%, 5%, 6%, 7%, or greater than 7%, wherein the range is inclusive and each number may be modified by the term “about”), the methods of the invention may still be carried out. In some embodiments, as discussed below, it may be preferable to use fresh fungi or fungi that is only partially dried.

In some preferred embodiments, the fungi will be only partially dried so the mushrooms retain some pliability (“pliably dried”). For example, in some exemplary embodiments, fungi may be dried with a fan for approximately 3 days to reach a partially dry state in preparation for extraction. If preparing the mushrooms for extraction by, for example, chopping, achieving this partially dry state will help minimize bruising to the flesh of the fungus when chopping. Minimizing the bruising of a fungus can ensure that active agent content is not degraded, as can occur when the flesh of certain fungi, for example psilocybin mushrooms, are bruised.

In some embodiments wherein drying is performed (120), drying may be completed at a maximum temperature of 50° C., including 49° C., 48° C., 47° C., 46° C., 45° C., 44° C., 43° C., 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., or less than 20° C. As it relates to performing the drying specifically, the fungi may be dried in a circulated air dehydration unit, a forced air food dryer, or any other such device capable of maintaining a substantially constant temperature set by a user, wherein heating is completed via conduction (direct heating), convection (heating via continuous currents of a gas or liquid), and/or radiation (heating via absorption of heat by a cooler body from a warmer body).

Additionally, drying may, in some embodiments, last from about 24 hours to about 120 hours, including 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56, hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, 72 hours, 73 hours, 74 hours, 75 hours, 76 hours, 77hours, 78 hours, 79 hours, 80 hours, 81 hours, 82 hours, 83 hours, 84 hours, 85 hours, 86 hours, 87 hours, 88 hours, 89 hours, 90 hours, 91 hours, 92 hours, 93 hours, 94 hours, 95 hours, 96 hours, 97 hours, 98 hours, 99 hours, 100 hours, 101 hours, 102 hours, 103 hours, 104 hours, 105 hours, 106 hours, 107 hours, 108 hours, 109 hours, 110 hours, 111 hours, 112 hours, 113 hours, 116 hours, 117 hours, 118 hours, 119 hours, and 120 hours wherein the range is inclusive and each value will be understood as being both modified and not modified by the term “about.”

In some embodiments, whether or not the fungi are dehydrated, they may be sealed in plastic bags or airtight containers and stored in darkness until processing. In such embodiments, the temperature at which the fungi are stored should correspond with how soon processing may take place. Generally, fungi are stored at between about −25° C. and about 3° C. However, but not to be bound by theory, storage at the warmer temperatures of the aforementioned range is only recommended if processing will imminently occur (such as within about 5 days) because prolonged storage at temperatures above 3° C. may result in a loss of potency and gradual destruction of the fungi by macroscopic and microscopic organisms.

In some embodiments, the fungi are prepared for extraction by being sliced (120). In another embodiment, the fungi are prepared by being pulled into pieces by hand. Acceptable slices or pieces of fungus may be about 1 cm to about 8 cm portions, including 1 cm, 2 cm, 3 cm, 4 cm, 5 cm, 6 cm, 7 cm, and 8 cm, wherein the range is inclusive and each value will be understood as being both modified and not modified by the term “about.”.

In some embodiments, the fungi are prepared for extraction by being pulverized to a powder (120). The fungi may be pulverized to a powder that may or may not be a substantially fine powder by a food processor, coffee grinder, blender, mortar and pestle, or similar device for, in some embodiments, between about 10 seconds, about 20 seconds, about 45 seconds, or about one minute, wherein the range is inclusive and each value will be understood as being both modified and not modified by the term “about.”

In an optional step (200), the fungi are subjected to quality assurance analyses prior to extraction. At this point, or at any point prior to preparation of the fungi, in some embodiments, a small batch of the prepared fungi may optionally be analyzed to determine whether the active agents are within safety and production specifications. In addition, the batch may or may not be analyzed for heavy metal and pesticide content. The analysis may be performed by any device known to those of skill capable of completing such an analysis with one non-limiting example being high performance liquid chromatography tandem mass spectrometry (“HPLC-MS/MS”). In some embodiments, the batch must include at least about 500 micrograms per gram of active agent to proceed with the method of the invention.

In some embodiments, a juice and a pH modulating ingredient, such as malic acid, are combined to obtain a solvent having a desired pH.

In some embodiments, the fungi, whether prepared or not, are combined with a solvent (140) having an acidic pH (i.e., a pH below 7), including a pH below 6.5, 6.0, 5.5, 5.0, 4.5, 4.0, 3.5, 3.0, 2.5, 2.0, 1.5, 1.0, and 0.5, including up to 0.0, wherein each value will be understood as being both modified and not modified by the term “about.” In some embodiments, the solvent has an acidic pH of greater than 0.0, 1.0, 1.5, 2.0, 2.5, 3.0, 3.5, 4.0, 4.5, 5.0, 5.5, 6.0, and 6.5, including up to 7.0. In some preferred embodiments, the pH of the solvent ranges from about 2.0 to about 6.0, or about 3.0 to about 5.0, or about 3.5 to 4.5, or about 3.5 to about 4.0.

In some embodiments, the pH of a juice selected for extraction is either increased or decreased by the addition of another ingredient having a pH sufficient to modulate the solvent pH, for example a stronger acid or a base (130). For example, apple cider vinegar or malic acid could be combined with apple juice to obtain a more acidic solvent.

In embodiments, the solvent consists of apple juice. The apple juice may be a filtered apple juice or an unfiltered apple juice. The apple juice may be a sweetener-free apple juice, a sugar-free apple juice, an apple juice with added sweetener, or an apple juice with added sugar. The apple juice may be made from concentrate, or may be not from concentrate. In embodiments the apple juice has added ascorbic acid (vitamin C). In embodiments, the apple juice may have additional ingredients, such as preservatives, such as calcium citrate or potassium phosphate.

In some embodiments the solvent is chilled prior to being combined with the fungus. In some embodiments the temperature of the solvent may be from about 15° C. to about 0° C. In a preferred embodiment, the temperature of the solvent may be from about 3° C. to about 5° C. Utilizing a chilled solvent, rather than a hot solvent (one above about 30° C.), may help to preserve certain active agents during the extraction process, thereby allowing for the production of more potent extracts.

In some embodiments, the solvent utilized for extraction may be one or more of apple juice, lime juice, lemon juice, grape juice, pineapple juice, orange juice, or grapefruit juice or any other juice known to one skilled in the art. In a preferred embodiment, the juice selected for extraction is apple juice due to its ability to increase palatability of active agents and reduce negative symptoms associated with consuming certain psychedelic or functional mushrooms, such as acid reflux, upset stomach, nausea, and vomiting.

In some embodiments, the fungi, whether prepared or not, are placed in a quantity of solvent, for example apple juice, sufficient to substantially cover the prepared mushrooms. The fungi and solvent may then be stirred to create a slurry (150).

It will be readily appreciated that, although the length of time for stirring may differ, the duration need only be that which is sufficient to mix the solution, such that the fungi and solvent form a slurry. So, the method of mixing may not be important, so long as mixing is adequately completed. Mixing may be done by hand, via utilization of a stirring utensil, such as a whisk, a spoon, or a spatula; using a stir plate, using an electronic hand mixer, using an electronic mixer having its own arm, or, in some embodiments, using an industrial vat made of a substantially solid material capable of holding the slurry of the invention and possessing at least one agitation means (such as, but not limited to, paddles or arms). All that is required is an apparatus or utensils capable of providing the necessary amount of agitation for the required amount of time.

In some embodiments, the slurry may be allowed to stand for an extraction period, which will be appreciated by one of skill to be a period of time sufficient to extract active agents from the prepared fungi. Acceptable extraction times may range from about 4 hours to about 240 hours, including 8 hours, 12 hours, 24 hours, 48 hours, 72 hours, 96 hours, 120 hours, 144 hours, 168 hours, 192 hours, 216 hours, and 240 hours, wherein the range is inclusive and each value will be understood as being both modified and not modified by the term “about.”

In some embodiments, the slurry will be placed in a temperature controlled environment for the extraction period. In some embodiments, the slurry will be placed in a chilled environment (160). In some embodiments, temperature controlled environments may be from about 15° C. to about 0° C., including 15° C., 14° C., 13° C., 12° C., 11° C., 10° C., 9° C., 8° C., 7° C., 6° C., 5° C., 4° C., 3° C., 2° C., 1° C., and 0° C., wherein the range is inclusive and each value will be understood as being both modified and not modified by the term “about.”

In some embodiments, performing the extraction in a chilled environment will prevent the destruction of the fungi by macroscopic and microscopic organisms, such as bacteria or mold, during the extraction period. Quantification of the destruction of the fungi or the presence of organisms such as bacteria or mold, may be according to known methods.

In some embodiments, performing the extraction in a chilled environment will prevent the degradation of one or more active agents from fungi compared to extraction methods which utilize hot environments for the extraction process, such as temperatures above about 30° C. Quantification of the active agents may be according to known methods.

In some embodiments the slurry may be stirred or agitated continuously (i.e., in intervals below 1 minute) throughout the extraction period.

In some embodiments, the slurry may be stirred or agitated periodically throughout the extraction period (210). In some embodiments the slurry may be stirred in intervals from about 1 minute to about 3 days. As it relates to stirring specifically, the slurry is stirred for between about 1 minute to about 180 minutes, including 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, or greater than 180 minutes, wherein the range is inclusive and each value may be modified by the term “about.”

In some embodiments, the stirring or agitation may be done at between 1 rpm and 2500 rpm, including about 1 rpm, 10 rpm, 25 rpm, 50 rpm, 100 rpm, 150 rpm, 200 rpm, 250 rpm, 300 rpm, 350 rpm, 400 rpm, 450 rpm, 500 rpm, 550 rpm, 600 rpm, 650 rpm, 700 rpm, 750 rpm, 800 rpm, 850 rpm, 900 rpm, 950 rpm, 1000 rpm, 1050 rpm, 1100 rpm, 1150 rpm, 1200 rpm, 1250 rpm, 1300 rpm, 1350 rpm, 1400 rpm, 1450 rpm, 1500 rpm, 1550 rpm, 1600 rpm, 1650 rpm, 1700 rpm, 1750 rpm, 1800 rpm, 1850 rpm, 1900 rpm, 1950 rpm, 2000 rpm, 2050 rpm, 2100 rpm, 2150 rpm, 2200 rpm, 2250 rpm, 2300 rpm, 2350 rpm, 2400 rpm, 2450 rpm, 2500 rpm, and values in between, wherein each value may be modified by the term “about.”

As it relates to time specifically, agitation may be completed for between about 1 minute, to at least 180 minutes—the specific amount of time required reflecting the given embodiment being practiced. Meaning, in some embodiments, the duration of time required for agitation may be based on a standard amount known for that given embodiment, such as but not limited to an embodiment wherein the suggested agitation time is 10 minutes. In other embodiments, agitation may be an iterative process wherein agitation ceases when the slurry forms a substantially homogeneous slurry. As it relates to the invention as disclosed herein, a slurry is characterized by substantially uniform dispersion of the fungal material within the solvent, which may be evidenced by the naked eye, and would be immediately apparent to one of skill. However, such uniform dispersion may additionally be determined electronically, using devices known to those of skill capable of determining sample variance.

An acceptable ratio of fungi to solvent will be appreciated by one skilled in the art to be that which allows for the extraction of active agents from the fungi. In some embodiments, the ratio of fungi, whether prepared or not, to solvent may be about 1 gram of fungi: 40 mL of solvent, such that 1 gram, 5 grams, 7 grams, 10 grams, 12 grams, 15 grams, 17 grams, 20 grams, 25 grams, 30 grams, 35 grams, 40 grams, 45 grams, 50 grams, 55 grams, 60 grams, 65 grams, 70 grams, 75 grams, 80 grams, 85 grams, 90 grams, 95 grams, or 100 grams of fungi may be combined with 40 mL, 80 mL, 280 mL, 400 mL, 480 mL, 600 mL, 680 mL, 800 mL, 1000 mL, 1200 mL, 1400 mL, 1600 mL, 2000 mL, 2200 mL, 2400 mL, 2600 mL, 2800 mL, 3000 mL, 3200 mL, 3400 mL, 3600 mL, 3800 mL, 4000 mL, respectively.

After the extraction period is complete, the slurry may be removed from the chilled environment (170). In some embodiments, the slurry is then filtered to remove solids (180). Any filter known to those of skill may be utilized, so long as the filter is capable of filtering out both large and small particles. In fact, in some embodiments, a plurality of filters may be used, wherein the pore size decreases with each additional filter. In such embodiments, at least two, at least three, at least four, at least five, or more than five filters may be utilized. This is particularly useful if certain particles of a given size are desired in the extract, or to be individually isolated.

In some embodiments, a cheesecloth or muslin cloth may be utilized as a filter to remove larger particles while glass wool may be used as a filter to remove smaller particles. In some embodiments, the extract may be collected in a flask, such as an Erlenmeyer flask. However, this is merely an exemplary, non-limiting embodiment. As would be apparent to one of skill, any such substantially solid container sufficient to contain the extract without leaking may be used for collection. In some embodiments, pressure is applied during the filtration process, such as with use of a fruit press, or manually by squeezing.

In other yet embodiments, the slurry may be drawn through the filter(s) by vacuum filtration. It will be appreciated that if the filter does not clog, filtration may take about 15 minutes; however, if the filter gets clogged, filtration may take from about 30 mins to about 60 mins, for example. In another example, the slurry may be placed in a nylon filter bag, such as a 75 micron filter bag, and slowly filtered by a filter press over 60 minutes, for example. In an optional embodiment, the extract is centrifuged to separate particulate matter and a supernatant.

In some embodiments, the pH of the extract may be adjusted (190) by adding a pH modulating ingredient (i.e., any ingredient capable of changing the pH of an extract). The pH may be increased from the pH of the extract by adding a base, or a neutral compound such as water. In embodiments, the base used to modulate the pH of the extract is calcium hydroxide, sodium bicarbonate, calcium acetate, magnesium hydroxide, or any other compound known to those of skill to be capable of raising the pH of the extract. Raising the pH of the extract after extraction may further reduce heartburn, nausea, upset stomach, acid reflux and other unwanted side effects associated with consuming psychedelic or functional mushrooms as well as acidic foods or beverages.

In some embodiments, the pH of the extract is about 3.8 (the approximate pH of apple juice). The pH of the extract may be increased by at least about 0.1 to about 10. In some embodiments the pH of the extract is increased above about 4.0, including 5.0, 6.0, 7.0, 8.0, 9.0, 10.0, 11.0, or higher, wherein the range is inclusive and each value will be understood as being both modified and not modified by the term “about.”

In some embodiments, the extract is concentrated. In some embodiments, the concentrating step comprises heating the extract. In some embodiments, the duration of heat exposure is from about 0.5 hours to about 6 hours, wherein the range is inclusive. In some embodiments, the duration of heat exposure is about 0.5 h, 1 h, 1.5 hours, 2 hours, 2.5 hours, 3 hours, 3.5 hours, 4 hours, 4.5 hours, 5 hours, 5.5 hours, or 6 hours. In some embodiments, the heat exposure exceeds 6 hours. In some embodiments, the extract is heated from about 75° C. to about 177° C. (200° F.-350° F.), including about 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C., 160° C., 161° C., 162° C., 163° C., 164° C., 165° C., 166° C., 167° C., 168° C., 169° C., 170° C., 171° C., 172° C., 173° C., 174° C., 175° C., 176° C., 177° C., and values in between, wherein each value is modified and not modified by the term “about.”

After filtration in Step 180, the extract may also be optionally centrifuged. In some such embodiments, the extract will be centrifuged to remove unwanted particles from the extract, such as fungal biomass. In embodiments wherein centrifuging is completed, it may take place for between about 1 and about 25 minutes, including 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, and values in between, wherein each value may be modified by the term “about;” at between about 2000 to about 6000 rpm, including about 2100 rpm, 2200 rpm, 2300 rpm, 2400 rpm, 2500 rpm, 2600 rpm, 2700 rpm, 2800 rpm, 2900 rpm, 3000 rpm, 3100 rpm, 3200 rpm, 3300 rpm, 3400 rpm, 3500 rpm, 3600 rpm, 3700 rpm, 3800 rpm, 3900 rpm, 4000 rpm, 4100 rpm, 4200 rpm, 4300 rpm, 4400 rpm, 4500 rpm, 4600 rpm, 4700 rpm, 4800 rpm, 4900 rpm, 5000 rpm, 5100 rpm, 5200 rpm, 5300 rpm, 5400 rpm, 5500 rpm, 5600 rpm, 5700 rpm, 5800 rpm, 5900 rpm, 6000 rpm, and values in between, wherein each value may be modified by the term “about.” In some embodiments, centrifuging may take place for about 15 minutes at about 4000 rpm at room temperature. In any such embodiments, if there is a resulting pellet of non-soluble and/or fibrous material, it is discarded. The supernatant is then collected.

In some embodiments, the extraction process may be completed multiple times (220) to increase the potency of the extract (i.e., the concentration of one or more active agents in the extract) without applying heat to the extract, which could degrade some of the active agents. For example, an extract may be used in place of juice for the extraction process, whereby fungi are combined with an extract containing one or more active agents from a prior extraction. The extraction process is then conducted in accordance with the methods disclosed herein.

In some exemplary embodiments, the fungus selected for extraction is a psilocybin mushroom, the solvent selected for extraction has a pH of about 3.0 to about 5.0, and the extraction is carried out at a temperature from about 1° C. to about 10° C. In some exemplary embodiments, the fungus obtained and selected for extraction is a psilocybin mushroom, the solvent selected for extraction is apple juice having a pH of about 3.8, and the extraction is carried out at a temperature of about 4° C.

In some exemplary embodiments, the fungus selected for extraction is a lion's mane mushroom, the solvent selected for extraction has a pH of about 3.0 to about 5.0, and the extraction is carried out at a temperature from about 1° C. to about 10° C. In some exemplary embodiments, the fungus obtained and selected for extraction is a lion's mane mushroom, the solvent selected for extraction is apple juice having a pH of about 3.8, and the extraction is carried out at a temperature of about 4° C.

In some exemplary embodiments, the fungus selected for extraction is a reishi mushroom, the solvent selected for extraction has a pH of about 3.0 to about 5.0, and the extraction is carried out at a temperature from about 1° C. to about 10° C. In some exemplary embodiments, the fungus obtained and selected for extraction is a reishi mushroom, the solvent selected for extraction is apple juice having a pH of about 3.8, and the extraction is carried out at a temperature of about 4° C.

In some exemplary embodiments, the fungus selected for extraction is a cordyceps mushroom, the solvent selected for extraction has a pH of about 3.0 to about 5.0, and the extraction is carried out at a temperature from about 1° C. to about 10° C. In some exemplary embodiments, the fungus obtained and selected for extraction is a cordyceps mushroom, the solvent selected for extraction is apple juice having a pH of about 3.8, and the extraction is carried out at a temperature of about 4° C.

In some exemplary embodiments, including the exemplary embodiments above using a psilocybin mushroom, lion's mane mushroom, reishi mushroom, and cordyceps mushroom, the extraction period is at least 4 hours, at least 8 hours, at least 16 hours, or at least 24 hours.

In embodiments, the extract is stored under light-protected conditions. In embodiments, the extract is stored in a green, amber, translucent, or opaque container. In embodiments, an opaque material, e.g., foil, e.g., aluminum foil, is used to cover the container holding the extract.

### D. EXTRACT COMPOSITIONS

Fungal extracts prepared in accordance with embodiments of the invention have multiple applications for the improvement of human health and wellbeing, including to reduce pain and treat pain disorders, to reduce and treat inflammation and inflammatory disorders, to benefit immunity and reduce or treat symptoms of psychological disorders, immune disorders, including autoimmune diseases and disorders, and for the general improvement of physical health and wellness including relaxation, enhanced cognitive function, and improvement in sleep, as illustrative and non-limiting examples.

In accordance with one embodiment of the invention, the extract may be prepared for ingestion. In some embodiments, the extract is prepared for ingestion by being mixed, combined, or otherwise formulated into an ice cube, popsicle, food, and beverage. In some embodiments, the extract, whether in its raw form of after being prepared for ingestion, contains at least one active agent in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg, as well as amounts within these ranges.

In some embodiments, the active agent is any of: an alkaloid, a saccharide, an amino acid, a vitamin, a mineral, and a lipid. In some embodiments, the active agent is any of: a terpene, a tryptamine, a peptide, a protein, and a polysaccharide. In some embodiments, the active agent is any of: psilocybin, psilocin, norpsilocin, baeocystin, norbaeocystin, aeruginascin, β-carbolines, triterpenoids, Hericenone A, Hericenone B, Hericenone C, Hericenone D, Hericenone E, Hericenone F, Hericenone G, Hericenone H, Hericenone I, Hericenone J, Hericenone K, 3HF, DLPE, Isohericerinol A, Hericerin, NDPIH, Erinacine A, Erinacine B, Erinacine C, Erinacine D, Erinacine E, Erinacine F, Erinacine G, Erinacine H, Erinacine I, Erinacine J, Erinacine K, Erinacine P, Erinacine Q, and Corallocin A, cordycepic acid, N-acetylgalactosamine, adenosine, ergosterol, ergosteryl esters, bioxanthracenes, hypoxanthine, acid deoxyribonuclease, polysaccharide, exopolysaccharide, chitinase, macrolides, cicadapeptins, myriocin, superoxide dismutase, protease, naphthoquinone, cordyheptapeptide, dipicolinic acid, fibrinolytical enzyme, lectin, cordymin, triterpene, steroid, and cordycepin.

In embodiments, where the extract contains a compound from lion's mane, such as a hericenone or erinacine extracted from Hericium erinaceum, and including any of Hericenone A, Hericenone B, Hericenone C, Hericenone D, Hericenone E, Hericenone F, Hericenone G, Hericenone H, Hericenone I, Hericenone J, Hericenone K, 3HF, DLPE, Isohericerinol A, Hericerin, NDPIH, Erinacine A, Erinacine B, Erinacine C, Erinacine D, Erinacine E, Erinacine F, Erinacine G, Erinacine H, Erinacine I, Erinacine J, Erinacine K, Erinacine P, Erinacine Q, and Corallocin A. In embodiments, where the extract contains a compound from cordyceps, such as cordycepin, cordycepic acid, N-acetylgalactosamine, adenosine, ergosterol, ergosteryl esters, bioxanthracenes, hypoxanthine, acid deoxyribonuclease, polysaccharide, exopolysaccharide, chitinase, macrolides, cicadapeptins, myriocin, superoxide dismutase, protease, naphthoquinone, cordyheptapeptide, dipicolinic acid, fibrinolytical enzyme, lectin, and cordymin. In embodiments, where the extract contains a compound from reishi, such as triterpene, polysaccharide, amino acid, mineral, and steroid. In some embodiments, where the extract contains a compound from a psilocybin mushroom, such as psilocybin, psilocin, norbaeocystin, baeocystin, aeruginascin, norpsilocin, and β-carbolines.

In some embodiments, the extract contains any of an active agent and an additional active ingredient that produces a therapeutic effect. In some embodiments, the therapeutic effect is an antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, entactogenic, empathogenic, entheogenic, psychedelic, sedative, or stimulant effect.

In some embodiments, one or more additional ingredients will be added to the extract to improve the taste, reduce nausea associated with consuming certain psychedelic and functional mushrooms, or provide additional medicinal benefits (280).

In some embodiments, at least one additional ingredient is selected to be combined with at least one extract containing one or more active agents from the following: a flavoring agent, sweetener, vitamin, herb, plant, and plant extract.

In some embodiments the extract is combined with at least one of the following: ginger, peppermint, spearmint, cinnamon, Vitamin C, chamomile, licorice root, cardamom, dandelion, marshmallow root, and slippery root.

In embodiments, the extract further comprises a therapeutically effective amount of an additional active ingredient. In embodiments, the additional active compound acts to increase therapeutic efficacy, provide additional therapeutic effects, decrease unwanted effects, increase stability or shelf-life, improve bioavailability, induce synergy, or alter pharmacokinetics or pharmacodynamics. In embodiments, the additional active compound is selected from the group consisting of: amino acids, antioxidants, anti-inflammatory agents, analgesics, antineuropathic and antinociceptive agents, antimigraine agents, anxiolytics, antidepressants, antipsychotics, anti-PTSD agents, immunostimulants, anti-cancer agents, antiemetics, orexigenics, antiulcer agents, antihistamines, antihypertensives, anticonvulsants, antiepileptics, bronchodilators, neuroprotectants, entactogens and empathogens, entheogens, psychedelics, nootropics, monoamine oxidase inhibitors, sedatives, stimulants, supplements, and vitamins.

In some embodiments, the extract may contain pharmaceutically acceptable excipients such as fillers, diluents, lubricants, surfactants, glidants, binders, dispersing agents, suspending agents, disintegrants, viscosity-increasing agents, film-forming agents, granulation aids, flavoring agents, sweetener, coating agents, solubilizing agents, and combinations thereof.

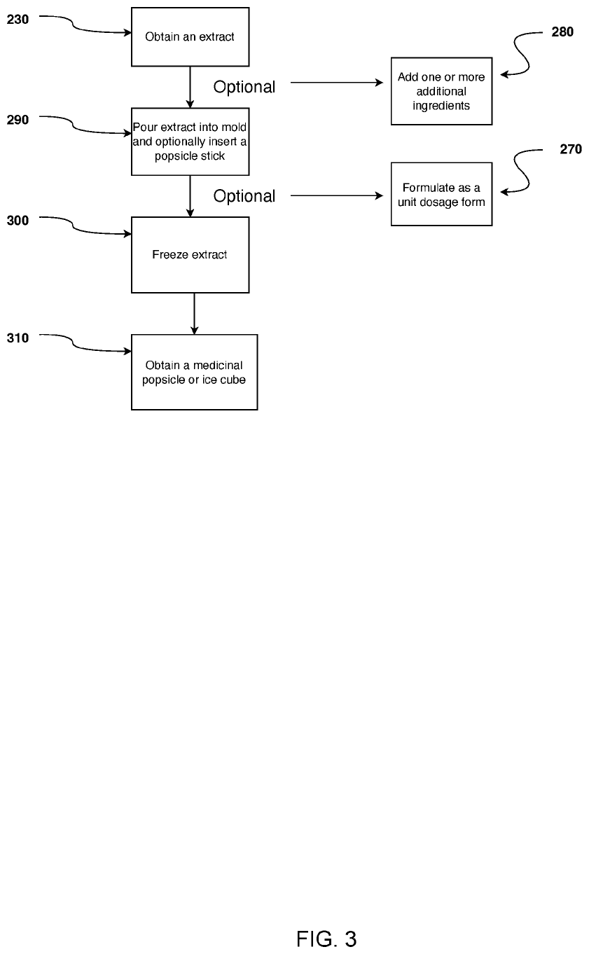

The present disclosure relates to formulations of medicinal popsicles and ice cubes (FIG. 3). The medicinal popsicles and ice cubes may be formulated by: obtaining an extract containing active agents using the methods disclosed herein (230), pouring the extract into a mold, optionally inserting a popsicle stick (290), and freezing the extract in the mold (300) until the extract solidifies into a medicinal popsicle or ice cube.

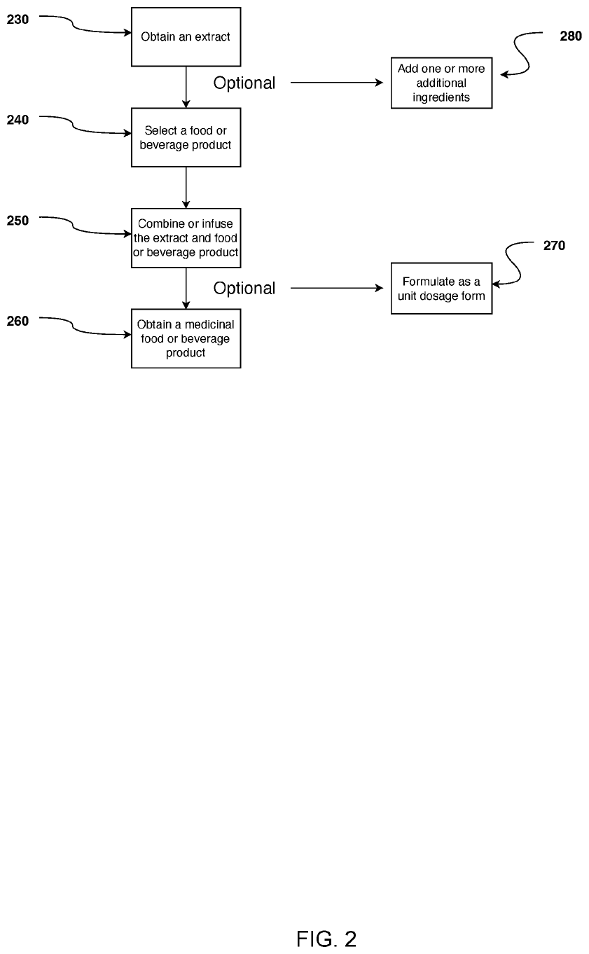

The present disclosure relates to formulations of medicinal food products (FIG. 2) in which an extract containing active agents, which was obtained using the methods disclosed herein (230) is infused, mixed, or otherwise combined with at least one food or beverage base resulting in a medicinal-infused food item. The extract may be optionally combined with one or more additional ingredients to further improve the taste or therapeutic value of the extract (280). In some embodiments, a food or beverage is selected to be combined with the extract to optimize taste or provide an additional therapeutic or nutritional benefit (240). Once selected, the food or beverage is infused, mixed or otherwise combined with the extract (250) for a period of time sufficient for the extract to combine with the food or beverage thereby creating the medicinal food or beverage product (260).

In some embodiments, the food or beverage item is processed through freeze and dehydration under a vacuum for extended periods, and is vacuum sealed with oxygen absorbers in place to provide a medicinal-infused food or beverage product that is uniformly dosed and is shelf stable for a prolonged period of time.

In some embodiments, one or more additional ingredients will be added to the food or beverage product to improve the taste, reduce nausea associated with consuming psychedelic or functional mushrooms, or provide additional medicinal benefits (280). In some embodiments, at least one additional ingredient is selected to be combined with at least one extract containing one or more active agents from the following: a flavoring agent, sweetener, vitamin, herb, and plant extract.

In some embodiments the food or beverage product is combined with at least one of the following: ginger, peppermint, spearmint, cinnamon, Vitamin C, chamomile, licorice root, cardamom, dandelion, marshmallow root, and slippery root.

In some embodiments, the extract is formulated in a unit dosage form. The term “unit dosage form” refers to a physically discrete unit suited as unitary dosages for the subject to be treated, each unit comprising a predetermined quantity of active agent calculated to produce the desired therapeutic effect(s), in association with a food product. Unit dosage forms are often used for ease of administration and uniformity of dosage. Unit dosage forms can contain a single or individual dose or unit, a sub-dose, or an appropriate fraction thereof (e.g., one half a “full” dose), of the fungal extract and optional additional ingredients administered. Unit dosage forms include food and beverage products, popsicles, and ice cubes.

Unit dosage forms may be formulated to provide “macrodoses” of active agents or “microdoses” as will be appreciated by one skilled in the art.

In some embodiments, a “microdose” is an amount of about one twentieth to about one fifth, and preferably about one tenth, the amount of a typical “macrodose.” For example, in some embodiments a typical macrodose of psilocybin is about 25 mg.

In some embodiments, the extract is formulated into a beverage capable of being administered in unit dosage form. For example, a single serving beverage containing a daily microdose or macrodose of one or more active agents. In some embodiments, the beverage will contain at least one active agent in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg, as well as amounts within these ranges.

In all embodiments herein that include dose amounts of less than about 1 mg, such dose amounts will be understood to include further specific dose amounts of about 0.5 mg or less, about 0.25 mg or less, about 0.1 mg or less, about 0.05 mg or less, about 0.005 mg or less, about 0.001 mg or less, and about 0.0005 mg or less, as well as amounts within these ranges.

In all embodiments herein that include dose amounts of at least about 1 mg or more, up to and including about 75 mg, such dose amounts will be understood to include further specific dose amounts of (with all such milligram dose amounts to be understood also to be preceded by the modifier “about”) 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, as well as amounts within these ranges.

In all embodiments herein that include dose amounts of 75 mg, as well as greater than 75 mg, including 100 mg, 150 mg, 200 mg, or greater than 200 mg, such dose amounts will be understood to include further specific dose amounts of 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, and 200 mg. Such dose amounts additionally will be understood to include amounts within these ranges, and in all such embodiments, a single dose moreover may be greater than 200 mg, including 225 mg, 250 mg, 300 mg, 400 mg, 500 mg, 600 mg, 700 mg, 800 mg, 900 mg, 1000 mg, or greater than 1000 mg.

In some embodiments, the extract is formulated into rehydrated fruit. In some embodiments, the rehydrated fruit is made by obtaining an extract according to the methods disclosed herein and combining the extract with dehydrated fruit for a period sufficient for the dehydrated fruit to absorb the extract, thereby rehydrating the fruit. The rehydrated fruit may be formulated to contain microdoses or macrodoses of one or more active agents. In some embodiments, the rehydrated fruit will contain at least one active agent in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg, as well as amounts within these ranges.

In some embodiments, the extract is formulated into an applesauce. In some embodiments, the applesauce is made by obtaining an extract according to the methods disclosed herein and combining the extract with prepared apples and a sweetening agent, such as sugar. In some embodiments, the apples are prepared by any of slicing, coring, peeling, and cooking. The applesauce may be formulated to contain microdoses or macrodoses of one or more active agents. In some embodiments, the applesauce will contain at least one active agent in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg, as well as amounts within these ranges.

In some embodiments, the extract is formulated into a jello. In some embodiments, the jello is made by obtaining an extract according to the methods disclosed herein; heating water; combining hot water with gelatin powder; mixing the gelatin powder and water mixture until the gelatin powder is dissolved; allowing the mixture to cool to room temperature; adding the extract; and chilling to form a jello. The jello may be formulated to contain microdoses or macrodoses of one or more active agents. In some embodiments, the jello will contain at least one active agent in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg, as well as amounts within these ranges.

In some embodiments, the extract is formulated into a gummy. The gummy may be formulated to contain microdoses or macrodoses of one or more active agents. In some embodiments, the gummy will contain at least one active agent in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg, as well as amounts within these ranges.

In some embodiments, the extract is formulated into juice. In some embodiments, the juice is made by obtaining an extract according to the methods disclosed herein; and combining the extract with a juice. The juice may be formulated to contain microdoses or macrodoses of one or more active agents. In some embodiments, the juice will contain at least one active agent in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg, as well as amounts within these ranges.

Formulations of food and beverage products may also comprise a frozen extract. (FIG. 3). Such frozen formulations may be obtained by pouring the extract into a mold (290). The mold containing the extract may then be placed into a temperature controlled environment having a temperature sufficient to freeze the extract (300). After an amount of time sufficient to freeze the extract contained in the mold has passed, a medicinal ice cube or popsicle may be obtained (310). In some embodiments, the extract will be combined with an additional ingredient prior to freezing (280).

In some embodiments, the extract is formulated into a popsicle capable of being administered in unit dosage form. In some embodiments, the popsicle is made by obtaining an extract according to the methods disclosed herein; pouring the extract into a mold; inserting a popsicle stick into the mold; and freezing the extract to form a popsicle. For example, a single serving beverage containing a daily microdose or macrodose of one or more active agents. In some embodiments the popsicle will contain at least one active agent in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg, as well as amounts within these ranges.

### E. METHODS OF ADMINISTRATION

In some embodiments, provided is a method of administering an extract or product of any one of the foregoing embodiments to produce a therapeutic effect. In some embodiments the therapeutic effect is any of antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, nootropic, entactogenic, empathogenic, entheogenic, euphoric, psychedelic, sedative, and stimulant effects.

In some embodiments, the method of administering an extract or product of any one of the foregoing embodiments reduces the negative side effect of consuming an active agent. In some embodiments, the negative side effect is any of heartburn, nausea, upset stomach, and vomiting.

In some embodiments, provided is a method of treating a medical condition in a mammal in need of such treatment, the method comprising administering the extract or product of any one of the foregoing embodiments. In some embodiments, the medical condition is a disorder linked to dysregulation or inadequate functioning of neurotransmission. In some embodiments, the disorder linked to dysregulation or inadequate functioning of neurotransmission is that of monoaminergic neurotransmission. In some embodiments, the disorder linked to dysregulation or inadequate functioning of neurotransmission is that of serotonergic, dopaminergic, or noradrenergic neurotransmission.

In some embodiments, the medical condition is a mental health disorder. In some embodiments, the mental health disorder is any of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, a substance use disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders.

In some embodiments, the mental health disorder is a disorder related to rigid modes of thinking. In some embodiments, the disorder related to rigid modes of thinking is anxiety, depression, addiction, an eating disorder, an alcohol or drug abuse or dependence disorder, OCD, or PTSD. In some embodiments, depression is major depressive disorder (MDD) or treatment-resistant depression (TRD). In some embodiments, anxiety is generalized anxiety disorder (GAD). In some embodiments, the substance use disorder is any of alcohol use disorder, nicotine dependency, opioid use disorder, sedative, hypnotic, or anxiolytic use disorder, stimulant use disorder, or tobacco use disorder.

In some embodiments, the medical condition is a neurodegenerative disorder. In some embodiments, the neurodegenerative disorder is any of Alzheimer's disease (AD), corticobasal degeneration (CBD), a form of dementia, Huntington's disease, Lytico-Bodig disease, mild cognitive impairment (MCI), a motor neuron disease, progressive supranuclear palsy (PSP), multiple sclerosis, Parkinson's disease, and traumatic brain injury (TBI).

In some embodiments, the medical condition is pain and/or a pain disorder. In some embodiments, the pain disorder is any of arthritis, allodynia, atypical trigeminal neuralgia, trigeminal neuralgia, somatoform disorder, hypoesthesia, hyperalgesia, neuralgia, neuritis, neurogenic pain, phantom limb pain, analgesia, anesthesia dolorosa, causalgia, sciatic nerve pain disorder, degenerative joint disorder, fibromyalgia, visceral disease, chronic pain disorders, headache disorders, migraine headaches, chronic cluster headaches, concussion headache, short-lasting unilateral neuralgiform headache attacks, chronic fatigue syndrome, complex regional pain syndrome, neurodystrophy, plantar fasciitis, or pain associated with cancer.

In some embodiments, the medical condition is inflammation and/or an inflammatory disorder. In some embodiments, the inflammatory disorder is characterized by any one or more of skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation, intestinal inflammation, neuroinflammation, and brain inflammation.

### F. EXAMPLES

### Example 1

Example 1 is an example of a method of extracting active agents from a fungus (FIG. 1).

The extraction process comprised the following steps:
1. About 78 grams (measured with a digital food scale) of fresh fruiting bodies from a psilocybin containing fungus, herein referred to as “mushrooms,” were obtained and selected for extraction (110).
2. The mushrooms were then prepared for extraction (120) by being sliced into pieces of about 1.0 inch to about 1.5 inches in length.
3. Next, about 18 oz of cold apple juice having a pH of about 3.78 to about 3.80 (measured with a pH meter) were obtained (130), and combined with the prepared mushrooms in a bowl (140) and stirred to form a slurry (150).
4. The bowl containing the slurry was then covered with a lid and placed in a refrigerator (160) having a temperature of about 38° F. to about 40° F. (measured with an external temperature gauge) for about 4 days (99 hours and 30 minutes).
5. The bowl was periodically opened and stirred (210) over the about 4 day period. After about 4 days, the bowl containing the slurry was removed from the refrigerator (170).
6. The slurry was then filtered to remove the mushroom material by pouring the slurry through a muslin cloth then twisting and squeezing out the remaining extraction liquid from the mushroom material to form an extract containing active agents (180).

The resulting extract was observed to be a golden to yellowish color and to be opaque, no longer having the translucent appearance of apple juice. The extract was tasted and noted to have acquired a distinct psilocybin flavor.

The extract was then used (230) to create ice cubes and popsicles (FIG. 3). The extract was poured into silicone molds (290) and placed into a freezer (300) having a temperature of about −6° F. (measured with an external temperature gauge) until the liquid extract solidified (310). The popsicles and ice cubes were removed from the freezer once solid for consumption.

The extract popsicles and ice cubes were consumed by two human volunteers. The first human volunteer consumed one extract popsicle and one extract ice cube and the second human volunteer consumed two extract popsicles. The human volunteers who had both had prior experience with psychedelic mushrooms noted mild psychedelic effects after consuming the extract ice cubes and/or popsicles. The first volunteer reported a mild stoning effect after consuming the popsicle and ice cube and said that it felt like he had consumed a recreational dose of psilocybin mushrooms. The second human volunteer first consumed only one extract popsicle and noted a small change in perception. The second human volunteer then consumed a second extract popsicle and reported visual distortions and a very pleasant feeling. The human volunteers also noted that consumption of the extract popsicles and ice cubes did not produce unpleasant side effects which often accompany the ingestion of psilocybin containing mushrooms, such as nausea, vomiting, and acid reflux.

### Example 2

Example 2 is another example of an embodiment of the invention.

The extraction process comprised the following steps:
1. About 28 grams (measured with a digital food scale) of dried Lion's Mane fruiting bodies, herein referred to as “mushrooms,” were obtained and selected for extraction.
2. The mushrooms were prepared for extraction by being broken up by hand into pieces of about 1.0 inch to about 1.5 in length.
3. Next, about 3 cups of cold apple juice having a pH of about 3.78 to about 3.80 (measured with a pH meter) were combined with the prepared mushrooms in a bowl and stirred to form a slurry.
4. The bowl containing the slurry was then covered with a lid and placed in a refrigerator having a temperature of about 38° F. to about 40° F. (measured with an external temperature gauge) for about 4 days (103 hours 52 minutes).
5. The bowl was periodically opened and stirred over the about 4 day period. After about 4 days, the bowl containing the slurry was removed from the refrigerator.
6. The slurry was then filtered to remove the mushroom material by pouring the slurry through a muslin cloth then twisting and squeezing out the remaining extraction liquid from the mushroom material to form an extract containing active agents.

The resulting extract was observed to be an auburn color and to be opaque, no longer having the translucent appearance of apple juice. The extract was tasted and noted to have acquired a distinct Lion's Mane mushroom flavor.

### Example 3

Example 3 is another example of an embodiment of the invention. The extraction process comprised the following steps:
1. About 14 grams (measured with a digital food scale) of dried Reishi fruiting bodies, herein referred to as “mushrooms,” were obtained and selected for extraction.
2. The mushrooms were prepared for extraction by being broken up by hand into pieces of about 1.0 inch to about 1.5 inches in length.
3. Next, about 2 cups of cold apple juice having a pH of about 3.78 to about 3.80 (measured with a pH meter) were combined with the prepared mushrooms in a bowl and stirred to form a slurry.
4. The bowl containing the slurry was then covered with a lid and placed in a refrigerator having a temperature of about 38° F. to about 40° F. (measured with external temperature gauge) for about 4 days (103 hours and 47 minutes). The bowl was periodically opened and stirred over the about 4 day period.
5. After about 4 days, the bowl containing the slurry was removed from the refrigerator. The slurry was then filtered to remove the mushroom material by pouring the slurry through a muslin cloth then twisting and squeezing out the remaining extraction liquid from the mushroom material to form an extract containing active agents.

The resulting extract was observed to be a light yellow color and to be partially translucent, though cloudier than apple juice. The extract was tasted and noted to have acquired a distinct bitter Reishi mushroom flavor and earthy smell.

### Example 4

Example 4 is another example of an embodiment of the invention. The extraction process comprised the following steps:
1. About 7 grams (measured with a digital food scale) of dried Cordyceps fruiting bodies, herein referred to as “mushrooms,” were obtained and selected for extraction.
2. The mushrooms were prepared for extraction by being broken up by hand into pieces of about 1.0 inch to about 1.5 inches in length.
3. Next, approximately 2 cups of cold apple juice having a pH of about 3.78 to about 3.80 (measured with a pH meter) were combined with the prepared mushrooms in a bowl and stirred to form a slurry.
4. The bowl containing the slurry was then covered with a lid and placed in a refrigerator having a temperature of about 38° C. to about 40° F. (measured with an external temperature gauge) for about 4 days (103 hours and 30 minutes).
5. The bowl was periodically opened and stirred over the about 4 day period. After about 4 days, the bowl containing the slurry was removed from the refrigerator.
6. The slurry was then filtered to remove the mushroom material by pouring the slurry through a muslin cloth then twisting and squeezing out the remaining extraction liquid from the mushroom material to form an extract containing active agents.

### Example 5

Example 5 is another example of an embodiment of the invention. The extraction process comprised the following steps:
1. About 12 grams (measured with a digital food scale) of dried fruiting bodies from psilocybin containing fungi, herein referred to as “mushrooms,” were obtained and selected for extraction.
2. The mushrooms were prepared for extraction by being broken up by hand into pieces of about 1.0 inch to about 1.5 inches in length.
3. Next, about 1.5 cups of cold apple juice having a pH of about 3.78 to about 3.80 (measured with a pH meter) were combined with the prepared mushrooms in a bowl and stirred to form a slurry.
4. The bowl containing the slurry was then covered with a lid and placed in a refrigerator having a temperature of about 38° F. to about 40° F. (measured with an external temperature gauge) for about 4 days.
5. The bowl was periodically opened and stirred over the 4 day period. After 4 days, the bowl containing the slurry was removed from the refrigerator.
6. The slurry was then filtered to remove the mushroom material by pouring the slurry through a muslin cloth then twisting and squeezing out the remaining extraction liquid from the mushroom material to form an extract containing active agents.

The resulting extract was observed to be a dark blue color and opaque. The extract was tasted and noted to have acquired a distinct psilocybin mushroom flavor and earthy smell.

### Example 6

Example 6 is another example of an embodiment of the invention. An extract containing active ingredients was combined with apples to create a medicinal applesauce, by a method comprising the following steps:
1. An extract was obtained according to the methods disclosed herein.
2. Three apples were peeled, cored, and sliced.
3. Next, the apples were combined with about ⅔ cup of sugar in a cooking pot.
4. Then, about 1.5 cups of Lion's Mane extract (enough to fully cover the apples) was poured into the cooking pot.
5. The mixture of apples, sugar, and extract was then heated to a boil in the cooking pot for from about 15 to about 20 minutes (until the apples were soft enough to mash with a fork).
6. The mixture was then removed from the heat and about ¼ teaspoon of cinnamon was added before allowing the mixture to cool.
7. Once cooled the mixture was placed in a food processor and chopped until slightly chunky.

### Example 7

Example 7 is another example of an embodiment of the invention (FIG. 2). An extract containing active ingredients was combined with blueberry jello mix to create medicinal jello, by a method comprising the following steps:
1. An extract was obtained according to the methods disclosed herein (230).
2. A package of blueberry jello mix was obtained (240) and the gelatin powder contained therein was poured into a mixing bowl.
3. Next, about 1.0 cup of water was placed in a cooking pot and heated on a stove until it boiled. The water was then removed from the heat and poured into the mixing bowl.
4. The mixing bowl was stirred until the powder dissolved.
5. The mixture of water, and gelatin powder was allowed to cool to room temperature before about 1.0 cup of psilocybin mushroom extract was added (250).
6. The mixture of water, gelatin powder, and psilocybin mushroom extract was then placed into a refrigerator until it stiffened into jello (260).

### Example 8

Example 8 is another example of an embodiment of the invention (FIG. 2). An extract comprising active ingredients was combined with dehydrated fruit to create medicinal rehydrated fruit, by a method comprising the following steps:
1. A frozen extract was obtained, which was made according to the methods disclosed herein by using fresh fruiting bodies from psilocybin containing fungi (230).
2. The frozen extract was put into a plastic measuring cup and covered with plastic wrap and then put into a refrigerator for about 2 days. After two days the mushroom extract had turned dark blue.
3. Pieces of dehydrated peaches and pineapples were obtained (240) and placed into a mixing bowl.
4. About 1.0 cup of psilocybin mushroom extract was poured into the mixing bowl with the dehydrated fruit (250).
5. The mixture of dehydrated fruit, and psilocybin mushroom extract was then placed into a refrigerator for about 15 hours.
6. After about 15 hours, the mixture was removed from the refrigerator and the dehydrated fruit was observed to be rehydrated, juicy and plump, having absorbed the psilocybin mushroom extract (260).

The foregoing description, for purposes of explanation, uses specific nomenclature to provide a thorough understanding of embodiments of the invention. However, it will be apparent to one skilled in the art that specific details are not required in order to practice embodiments of the invention. Thus, the foregoing description of specific embodiments of the invention is presented for purposes of illustration and description. It is not intended to be exhaustive or to limit the invention to the precise forms disclosed; obviously, many modifications and variations are possible in view of the above. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the invention shall be defined solely by the following claims and their equivalents.

The invention claimed is:

1. A method of extracting one or more active agents from a psychedelic or functional mushroom, the method comprising:
a. obtaining a fungus comprising one or more active agents;
b. preparing the fungus for extraction;
c. obtaining a solvent for extraction;
d. adding the fungus to the solvent and stirring to form a slurry;
e. chilling the slurry for an extraction period; and
f. straining the fungal biomass from the slurry to obtain the extract.

2. The method of claim 1, wherein the solvent is obtained by combining one or more juices with one or more pH modulating ingredients.

3. The method of claim 2, wherein the one or more pH modulating ingredients are any of acetic acid, malic acid, apple cider vinegar, and water.

4. The method of claim 2, wherein the one or more juices and the one or more pH modulating ingredients are combined to obtain a solvent with a desired pH level.

5. The method of any of claims 1-4, wherein the solvent does not include any of citric juice and citric acid.

6. The method of any of claims 1-4, wherein the solvent does not include any of lemon juice, lime juice, and orange juice.

7. The method of any of claims 1-4, wherein the solvent has a pH of between about 3.0 and 4.0.

8. The method of any of claims 1-4, wherein the solvent has a pH of greater than 3.5.

9. The method of any of claims 1-4, wherein the slurry is continuously stirred throughout the extraction period.

10. The method of any of claims 1-4, wherein the slurry is periodically stirred throughout the extraction period.

11. The method of claim 10, wherein the slurry is periodically stirred separated by intervals in between without stirring of about 1 minute to about 3 days.

12. The method of any of claims 1-4, wherein the slurry is stirred during the extraction period for at least one stirring period.

13. The method of claim 12, wherein the stirring period is from about 1 minute to about 180 minutes.

14. The method of any of claims 1-4, wherein the extraction period is at least about 4 hours.

15. The method of any of claims 1-4, wherein the extraction period is for between about 4 to about 130 hours.

16. The method of any of claims 1-4, wherein the extraction period is for between about 12 to about 120 hours.

17. The method of any of claims 1-4, wherein the extraction period is for between about 48 to about 110 hours.

18. The method of any of claims 1-4, wherein the extraction period is for between about 60 to about 96 hours.

19. The method of any of claims 1-4, wherein preparing the fungus for extraction includes drying the fungus.

20. The method of claim 19, wherein the fungus is dried for about 1 day to about 10 days.

21. The method of claim 19, wherein the fungus is cracker dry.

22. The method of claim 19, wherein the fungus is pliably dried.

23. The method of claim 22, wherein the fungus is pliably dried by drying for about 3 days.

24. The method of claim 19, wherein the fungus is dried by using any of a fan, a heat source, and a dehydrator.

25. The method of any of claims 1-4, wherein preparing the fungus for extraction includes any one or more of chopping, grinding, and pulverizing the fungus.

26. The method of any of claims 1-4, wherein stirring is completed by a utensil, such as a spoon, a fork, a knife, and/or a chopstick.

27. The method of any of claims 1-4, wherein stirring is completed utilizing an electric stirring device, such as an electric stick blender.

28. The method of any of claims 1-4, wherein the slurry is chilled for the extraction period in a refrigerator.

29. The method of any of claims 1-4, wherein the slurry is chilled at a temperature from about 35° F. and about 45° F.

30. The method of any of claims 1-4, wherein the straining is completed by any of a coffee filter, a cheesecloth, and a muslin cloth.

31. The method of any of claims 1-4, wherein the fungus is one or more species within a fungal genera selected from Psilocybe, Agaricus, Pleurotus, Lentinula, Auricularia, Volvariella, Flammulina, Tremella, Hypsizygus, Stropharia, Cyclocybe, Hericium, Phallus, Boletus, Calbovista, Calvatia, Cantharellus, Craterellus, Clitocybe, Cortinarius, Grifola, Macrolepiota, Gyromitra, Hydnum, Lactarius, Morchella, Tricholoma, Tuber, Trametes, Aleuria, Armillaria, Calocybe, Chroogomphus, Clavariaceae, Clavulinaceae, Coprinus, Cortinarius, Cyttaria, Fistulina, Flammulina, Hygrophorus, Kalaharituber, Laetiporus, Leccinum, Macrolepiota, Marasmius, Polyporus, Pseudohydnum, Ramariaceae, Ganoderma, Rhizopogon, Russula, Sparassis, Suillus, Coprinopsis, Lepista, Morchella, Chaga, Cordyceps, Verpa, Copelandia, Galerina, Gymnopilus, Inocybe, Panaeolus, Pholiotina, Inonotus, and Pluteus.

32. The method of any of claims 1-4, wherein the fungus is a psilocybin mushroom species within a fungal genera selected from a group consisting of Copelandia, Conocybe, Gymnopilus, Galerina, Inocybe, Panaeolus, Pholiotina, Pluteus, and Psilocybe.

33. The method of any of claims 1-4, wherein the fungus is a lion's mane mushroom.

34. The method of any of claims 1-4, wherein the fungus is a cordyceps mushroom.

35. The method of any of claims 1-4, wherein the fungus is a reishi mushroom.

36. An extract of a fungus obtained by any of the foregoing methods comprising at least one active agent from a mushroom.

37. The extract of claim 36, wherein the extract comprises an active agent from a cordyceps mushroom.

38. The extract of claim 37, wherein the active agent from a cordyceps mushroom is any of cordycepin, cordycepic acid, N-acetylgalactosamine, adenosine, ergosterol, an ergosteryl ester, a bioxanthracene, hypoxanthine, a macrolide, a cicadapeptin, myriocin, superoxide dismutase, naphthoquinone, cordyheptapeptide, dipicolinic acid, a fibrinolytic enzyme, and cordymin.

39. The extract of claim 36, wherein the extract comprises an active agent from a lion's mane mushroom.

40. The extract of claim 39, wherein the active agent from a lion's mane mushroom is any of Hericenone A, Hericenone B, Hericenone C, Hericenone D, Hericenone E, Hericenone F, Hericenone G, Hericenone H, Hericenone I, Hericenone J, Hericenone K, 3HF, DLPE, Isohericerinol A, Hericerin, NDPIH, Erinacine A, Erinacine B, Erinacine C, Erinacine D, Erinacine E, Erinacine F, Erinacine G, Erinacine H, Erinacine I, Erinacine J, Erinacine K, Erinacine P, Erinacine Q, and Corallocin A.

41. The extract of claim 36, wherein the extract comprises an active agent from a psilocybin mushroom.

42. The extract of claim 41, wherein the psilocybin mushroom is a species within a fungal genera selected from a group consisting of Copelandia, Conocybe, Gymnopilus, Galerina, Inocybe, Panaeolus, Pholiotina, Pluteus, and Psilocybe.

43. The extract of claim 41, wherein the active agent from a psilocybin mushroom is any of psilocybin, psilocin, norbaeocystin, baeocystin, aeruginascin, norpsilocin, and a β-carboline.

44. The extract of claim 36, wherein the extract comprises an active agent from reishi.

45. The extract of claim 44, wherein the active agent from a reishi is any of polysaccharides, triterpenoids, ganodermic acids, lucidenic acids, beta glucans, proteins, amino acids, ganoderic acids, ganodermanontriol, ganodermadiol, palmitic acid, ergosterol, enzymes, steroids, sterols, nucleotides, fatty acids, vitamins and minerals.

46. The extract of any of claims 36-45, wherein a base is added to the extract to increase the pH of the extract.

47. The extract of claim 46, wherein the base is any of calcium hydroxide, sodium bicarbonate, calcium acetate, and magnesium hydroxide.

48. The extract of any of claims 36-45, wherein the extract further comprises one or more additional ingredients selected from any of: ginger, peppermint, spearmint, cinnamon, Vitamin C, chamomile, licorice root, cardamom, dandelion, marshmallow root, and slippery root.

49. The extract of any of claims 36-45, wherein the extract is formulated in a unit dosage form.

50. The extract of any of claims 36-45, wherein the extract comprises a plurality of doses.

51. The extract of any of claims 36-45, wherein the potency of the active agent is less than about 0.05%.

52. The extract of any of claims 36-45, wherein the potency of the active agent is at least about 0.05%.

53. The extract of any of claims 36-45, wherein the potency of the active agent from about 0.05% to about 2.0%.

54. The extract of any of claims 36-45, wherein the active agent is from a psilocybin mushroom, and the potency of the active agent is at least about 0.05%, or from about 0.05% to about 2.0%.

55. The extract of any of claims 36-45, wherein the active agent is from a lion's mane mushroom, and the potency of the active agent is at least about 0.05%, or from about 0.05% to about 2.0%.

56. The extract of any of claims 36-45, wherein the active agent is from a cordyceps mushroom, and the potency of the active agent is at least about 0.05%, or from about 0.05% to about 2.0%.

57. The extract of any of claims 36-45, wherein the active agent is from a reishi mushroom, and the potency of the active agent is at least about 0.05%, or from about 0.05% to about 2.0%.

58. A product made with the extract of any of claims 36-45, wherein the product is prepared for ingestion.

59. The product of claim 58, wherein the product is prepared for ingestion as a popsicle, made by pouring the extract into a mold, inserting a popsicle stick into the mold, and freezing the extract.

60. The product of claim 58, wherein the product is prepared for ingestion as an ice cube, made by pouring the extract into a mold and freezing the extract.

61. The product of claim 58, wherein the product is prepared for ingestion as a medicinal food or beverage product, made by selecting a food or beverage product, and combining the food or beverage product with the extract.

62. The product of claim 58, wherein the product is prepared for ingestion as an apple sauce, made by preparing at least one apple and combining the prepared apple(s) with the extract and optionally with a quantity of a sweetening agent.

63. The product of claim 58, wherein the product is prepared for ingestion as a jello, made by heating water until hot, combining the hot water with gelatin powder, mixing the gelatin powder and water until the gelatin powder is dissolved in the water forming a mixture, allowing the mixture to cool to room temperature, adding the extract to the mixture, and chilling the mixture until it forms the jello.

64. The product of claim 58, wherein the product is prepared for ingestion by being formulated as a rehydrated fruit, made by combining the extract with dehydrated fruit and allowing the dehydrated fruit to rehydrate by absorbing the extract.

65. The product of claim 58, wherein the product is prepared for ingestion as a juice made by combining the extract with at least one juice.

66. The product of any of claims 58-65, wherein the product further comprises at least one additional ingredient.

67. The product of claim 66, wherein the additional ingredient is any of ginger, peppermint, spearmint, cinnamon, Vitamin C, chamomile, licorice root, cardamom, dandelion, marshmallow root, and slippery root.

68. The product of any of claims 58-65, wherein the product does not produce at least one negative symptom selected from any of nausea, heartburn, stomach pain, and vomiting.

69. The product of any of claims 58-65, wherein the product enhances any of mood, cognition, relaxation, wellbeing, and sleep.

70. The product of any of claims 58-65, wherein the product elicits a therapeutic effect.

71. The product of any of claims 58-65, wherein the therapeutic effect is any of antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, nootropic, entactogenic, empathogenic, entheogenic, euphoric, psychedelic, sedative, and stimulant effects.

72. The product of any of claims 58-65 for use to treat a medical condition in a human.

73. Use of the product of claim 72, wherein the medical condition is any of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, a substance use disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders.

74. Use of the product of claim 72, wherein the medical condition is a mental health disorder related to rigid modes of thinking.

75. Use of the product of claim 72, wherein the disorder related to rigid modes of thinking is any of anxiety, depression, addiction, an eating disorder, an alcohol or drug abuse or dependence disorder, OCD, or PTSD. In some embodiments, depression is major depressive disorder (MDD) or treatment-resistant depression (TRD). In some embodiments, anxiety is generalized anxiety disorder (GAD). In some embodiments, the substance use disorder is any of alcohol use disorder, nicotine dependency, opioid use disorder, sedative, hypnotic, or anxiolytic use disorder, stimulant use disorder, and tobacco use disorder.

76. Use of the product of claim 72, wherein the medical condition is a neurodegenerative disorder.

77. Use of the product of claim 72, wherein the neurodegenerative disorder is any of Alzheimer's disease (AD), corticobasal degeneration (CBD), a form of dementia, Huntington's disease, Lytico-Bodig disease, mild cognitive impairment (MCI), a motor neuron disease, progressive supranuclear palsy (PSP), multiple sclerosis, Parkinson's disease, and traumatic brain injury (TBI).

78. Use of the product of claim 72, wherein the medical condition is pain or a pain disorder.

79. Use of the product of claim 72, wherein the pain disorder is any of arthritis, allodynia, atypical trigeminal neuralgia, trigeminal neuralgia, somatoform disorder, hypoesthesia, hyperalgesia, neuralgia, neuritis, neurogenic pain, phantom limb pain, analgesia, anesthesia dolorosa, causalgia, sciatic nerve pain disorder, degenerative joint disorder, fibromyalgia, visceral disease, chronic pain disorders, headache disorders, migraine headaches, chronic cluster headaches, concussion headache, short-lasting unilateral neuralgiform headache attacks, chronic fatigue syndrome, complex regional pain syndrome, neurodystrophy, plantar fasciitis, and pain associated with cancer.

80. Use of the product of claim 72, wherein the medical condition is inflammation or an inflammatory disorder.

81. Use of the product of claim 72, wherein the inflammatory disorder is characterized by any one or more of skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation, intestinal inflammation, neuroinflammation, and brain inflammation.

82. The product of any of claims 58-65 for use to enhance any of mood, cognition, relaxation, wellbeing, and sleep.

83. The product of any of claims 58-65, formulated in a unit dosage form.

84. The product of any of claims 58-65, comprising a plurality of doses.

85. The product of any of claims 58-65, comprising a single dose.

86. The product of any of claims 58-65, comprising an active agent from a mushroom in an amount so that a single dose of the active agent is about 1 mg or less including a dose of about 0.5 mg or less, about 0.25 mg or less, about 0.1 mg or less, about 0.05 mg or less, about 0.005 mg or less, about 0.001 mg or less, and about 0.0005 mg or less.

87. The product of any of claims 58-65, comprising an active agent from a mushroom in an amount so that a single dose is at least about 1 mg to about 1000 mg or more.

88. The product of claim 87, wherein the active agent is from a psilocybin mushroom in an amount so that a single dose is from about 1 mg to about 70 mg.

89. The product of claim 88, wherein the active agent is psilocybin in an amount so that a single dose is from about 1 mg to about 70 mg.

90. The product of claim 88, wherein the active agent is psilocin in an amount so that a single dose is from about 1 mg to about 70 mg.

91. The product of claim 87, wherein the active agent is from a lion's mane mushroom in an amount so that a single dose is from about 20 mg to about 500 mg.

92. The product of claim 87, wherein the active agent is from a cordyceps mushroom in an amount so that a single dose is from about 20 mg to about 500 mg.

93. The product of claim 87, wherein the active agent is from a reishi mushroom in an amount so that a single dose is from about 20 mg to about 500 mg.

94. The product of claim 84, wherein the product is formulated to comprise a plurality of macrodoses.

95. The product of claim 84, wherein the product is formulated to comprise a plurality of microdoses.

96. A method of using the product of any of claims 58-65, to elicit a desired effect in a subject, the method comprising: obtaining an amount of the product capable of producing the desired effect, and administering the product to the subject.

97. The method of claim 96, wherein the desired effect is a therapeutic effect.

98. The method of claim 97, wherein the therapeutic effect is any of antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, nootropic, entactogenic, empathogenic, entheogenic, euphoric, psychedelic, sedative, and stimulant effects.

99. The method of claim 96, wherein the desired effect is a psychedelic effect.

100. The method of claim 96, wherein the desired effect is an enhancement in any of mood, cognition, relaxation, wellbeing, and sleep.

101. The method of claim 96, wherein the desired effect is any of improving health, delaying aging, increasing life expectancy, increasing creativity, increasing focus, and increasing mental acuity.

102. A method of using the product of any of claims 58-65 to treat a medical condition.

103. The method of claim 102, wherein the medical condition is any of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, a substance use disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders.

104. The method of claim 102, wherein the medical condition is a mental health disorder related to rigid modes of thinking.

105. The method of claim 104, wherein the disorder related to rigid modes of thinking is any of anxiety, depression, addiction, an eating disorder, an alcohol or drug abuse or dependence disorder, OCD, or PTSD. In some embodiments, depression is major depressive disorder (MDD) or treatment-resistant depression (TRD). In some embodiments, anxiety is generalized anxiety disorder (GAD). In some embodiments, the substance use disorder is any of alcohol use disorder, nicotine dependency, opioid use disorder, sedative, hypnotic, or anxiolytic use disorder, stimulant use disorder, and tobacco use disorder.

106. The method of claim 102, wherein the medical condition is a neurodegenerative disorder.

107. The method of claim 106, wherein, the neurodegenerative disorder is any of Alzheimer's disease (AD), corticobasal degeneration (CBD), a form of dementia, Huntington's disease, Lytico-Bodig disease, mild cognitive impairment (MCI), a motor neuron disease, progressive supranuclear palsy (PSP), multiple sclerosis, Parkinson's disease, and traumatic brain injury (TBI).

108. The method of claim 102, wherein the medical condition is pain or a pain disorder.

109. The method of claim 108, wherein the pain disorder is any of arthritis, allodynia, atypical trigeminal neuralgia, trigeminal neuralgia, somatoform disorder, hypoesthesia, hyperalgesia, neuralgia, neuritis, neurogenic pain, phantom limb pain, analgesia, anesthesia dolorosa, causalgia, sciatic nerve pain disorder, degenerative joint disorder, fibromyalgia, visceral disease, chronic pain disorders, headache disorders, migraine headaches, chronic cluster headaches, concussion headache, short-lasting unilateral neuralgiform headache attacks, chronic fatigue syndrome, complex regional pain syndrome, neurodystrophy, plantar fasciitis, and pain associated with cancer.

110. The method of claim 102, wherein medical condition is inflammation and/or an inflammatory disorder.

111. The method of claim 110, wherein the inflammatory disorder is characterized by any one or more of skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation, intestinal inflammation, neuroinflammation, and brain inflammation.

112. The method of any of claims 102-111, wherein the method does not cause a negative side effect associated with the consumption of psychedelic or functional mushrooms, or the ingestion of acidic foods or acidic beverages.

113. The method of any of claims 112, wherein the negative side effect is any of heartburn, nausea, upset stomach, and vomiting.

114. The method of any of claims 102-111, wherein the product is administered together with one or more sessions of psychotherapy.
